(19) United States
(12) Patent Application Publication
(10) Pub. No.: US 2022/0056499 A1
(43) Pub. Date: Feb. 24, 2022

(54) AGERITIN AS BIOINSECTICIDE AND METHODS OF GENERATING AND USING IT

(71) Applicant: SENCKENBERG GESELLSCHAFT FUER NATURFORSCHUNG, FRANKFURT (DE)

(72) Inventors: FLORIAN HENNICKE, FRANKFURT (DE); MARKUS KUENZLER, EHRENDINGEN (CH); ANNAGELDI TAYYROV, ZUERICH (CH); PETER LUETHY, FELDMEILEN (CH)

(21) Appl. No.: 17/416,798

(22) PCT Filed: Dec. 18, 2019
(86) PCT No.: PCT/EP2019/086004

§ 371 Date: Jun. 21, 2021

(30) Foreign Application Priority Data
Dec. 21, 2018 (EP) 18215370.0

(51) Int. Cl. C12P 21/02; C12N 15/82; A01N 63/50; C12N 9/88; C12N 15/80; C07K 14/415; A01P 7/04

(52) U.S. Cl. CPC C12P 21/02; C12N 15/8286; A01N 63/50; C12Y 406/01; C12N 15/80; C07K 14/415; A01P 7/04; C12N 9/88

(57) ABSTRACT

The present invention relates to the fungal protein ageritin, a nucleic acid molecule encoding said protein, host cells expressing the protein and/or the nucleic acid molecule and a plant or fungus expressing the protein and/or the nucleic acid molecule and/or comprising such host cells. The present invention further relates to using the fungal protein ageritin, the nucleic acid molecule encoding it, the host cell expressing it and/or the plant as bioinsecticide(s). The present invention further relates to a bioinsecticide composition.

The present invention relates to the fungal protein ageritin, a nucleic acid molecule encoding said protein, host cells expressing the protein and/or the nucleic acid molecule and a plant or fungus expressing the protein and/or the nucleic acid molecule and/or comprising such host cells. The present invention further relates to using the fungal protein ageritin, the nucleic acid molecule encoding it, the host cell expressing it and/or the plant as bioinsecticide(s). The present invention further relates to a bioinsecticide composition.

### BACKGROUND OF THE INVENTION

Fungi produce a variety of defense proteins against antagonists. One type of defense proteins are toxic ribonucleases also called ribotoxins. These toxins cleave a single phosphodiester bond within the universally conserved sarcin-ricin loop (SRL) of ribosomes and consequently inhibit protein synthesis.

Fungi are exposed to a large diversity of antagonists in their environment. To protect themselves, fungi mainly rely on (bio)chemical defense mediated by secondary metabolites, peptides and proteins (see e.g. Lacadena et al., 2007). They interfere with essential biological processes or structures within the target organisms. Ribosomes are essential molecular machineries present in all living cells making them ideal targets for defense proteins. Previous studies have revealed three main classes of proteins with ribonucleolytic activity towards ribosomal RNA. The first class comprises classical ribonucleases (RNases) that cleave any phosphodiester bond between ribonucleotides. These RNases have a rather low specificity for ribosomal RNAs and include non-toxic RNases. The second class of proteins is represented by ribosome inactivating proteins (RIPs) that act on ribosomes. RIPs are N-glycosidases that depurinate a specific adenine residue located in the highly conserved sarcin-ricin loop (SRL) of the large subunit of the eukaryotic and prokaryotic ribosomes. The depurination of the adenine disrupts the binding of the translation elongation factors, inhibiting the protein synthesis and leading to the death of the cells. High effectiveness and specificity makes RIPs a part of the defense systems of many organisms including bacteria (Endo et al., 1988), algae (Sawa et al., 2016), fungi (Yao et al., 1998) and plants (Bolognesi et al., 2016).

The third class of proteins with ribonucleolytic activity towards rRNA are called ribotoxins. They are small sized (10-20 kDa) and highly toxic (Lacadena et al., 2007; Herrero-Galan et al., 2009; Olombrada et al., 2013). Ribotoxins are highly specific fungal endonucleases that cleave a single phosphodiester bond at a universally conserved GAGA tetraloop of the SRL loop. Similar to RIPs, the damage of the loop inhibits binding of translation elongation factors and thus protein biosynthesis, ultimately leading to the death of the target cells (Correll et al., 1998). Until recently, fungal ribotoxins were exclusively known from members of the phylum Ascomycota. Two of the best-studied ribotoxins are α-sarcin (Perez-Canadillas et al., 2000) and restrictocin (Yang et al., 1996) produced by Aspergillus giganteus and Aspergillus restrictus, respectively. Hirsutellin A (Herrero-Galan et al., 2008) from the fungal pathogen of mites, Hirsutella thompsonii, and anisoplin (Olombrada et al., 2017) from the entomopathogenic fungus Metarhizium anisopliae are other, recently discovered ribotoxins. Lately, Landi et al. (2017) purified a protein, named ageritin, with ribonucleolytic activity towards rRNA from the commercially produced edible mushroom Agrocybe (Cyclocybe) aegerita, a member of the phylum Basidiomycota. In addition to the RNase activity, the authors demonstrated cytotoxicity and cell death promoting effects of ageritin against tumor cell lines of the human central nervous system (CNS).

Tropical mosquito species like the yellow fever mosquito Aedes aegypti are important disease vectors for some of the most important infectious diseases globally, including severe arboviral diseases such as yellow fever, chikungunya, dengue or zika. Stopping the development from larvae into adult mosquitos by larviciding (killing of their larvae) can efficiently abolish the establishment and spreading of stable populations by such mosquitos and can hence minimize the risk of disease transmission. Larviciding is currently done by introducing the biopesticide Bacillus thuringiensis israelensis (Bti) into bodies of standing water as potential sites of egg laying and larval hatching. However, this practice is potentially at risk of becoming ineffective as different genetic resistance mechanisms by Ae. aegypti and other mosquitos such as the pestiferous Culex quinquefasciatus have been reported (Suter et al., 2017).

However, this practice is potentially at risk of becoming ineffective as different reports on Bti resistance development by Ae. aegypti and other mosquitos such as the pestiferous Culex quinquefasciatus have been published (see e.g. Suter et al. 2017). In addition, the increasing spread of invasive Aedes mosquitoes leads to emergence of arboviral diseases such as dengue and chikungunya in more temperate regions (Barzon, 2018). For example, Ae. albopictus has invaded large parts of southern Europe. This insect is an efficient vector of dengue, chikungunya and zika (Kampen et al., 2017).

Also, from a broader perspective, insecticide resistance to currently available insecticides is a key challenge. Protective interventions such as insecticide-treated bed nets and indoor residual spraying become more and more ineffective.

In conclusion, there is a need in the art for developing novel means and methods for (biological) insecticides, and (biological) pesticides or compositions thereof.

### SUMMARY OF THE INVENTION

According to the present invention this object is solved by providing a protein selected from

a protein comprising an amino acid sequence of SEQ ID NO. 1 or an amino acid sequence of SEQ ID NO. 3, or an amino acid sequence having at least 60% sequence identity, preferably at least 70% sequence identity, more preferably at least 80% or 81% sequence identity to the amino acid sequence of SEQ ID NO. 1 or SEQ ID NO. 3, and/or
a protein encoded by a nucleotide sequence of SEQ ID NO. 2 or SEQ ID NO. 4, or a nucleotide sequence having at least 60% sequence identity, preferably 70% sequence identity, more preferably 80% or 81% sequence identity to the nucleotide sequence of SEQ ID NO. 2 or SEQ ID NO. 4.

According to the present invention this object is solved by providing a nucleic acid molecule, comprising a nucleotide sequence of SEQ ID NO. 2 or SEQ ID NO. 4 or a nucleotide sequence having at least 60% sequence identity, preferably 70% sequence identity, more preferably 80% or 81% sequence identity to the nucleotide sequence of SEQ ID NO. 2 or SEQ ID NO. 4.

According to the present invention this object is solved by providing a host cell, containing a nucleic acid molecule of the present invention and/or expressing the protein of the present invention.

According to the present invention this object is solved by providing a recombinant protein obtained from a host cell of the present invention.

According to the present invention this object is solved by providing a plant or fungus, containing a nucleic acid molecule according the present invention and/or expressing the protein of the present invention and/or comprising host cell(s) of the present invention.

According to the present invention this object is solved by using
the protein of the present invention, as defined herein,
the nucleic acid molecule of the present invention, as defined herein,
the host cell of the present invention, as defined herein, and/or
the plant or the fungus of the present invention, as defined herein,
as biological insecticide (bioinsecticide).

According to the present invention this object is solved by providing a (bio)insecticide or (bio)pesticide composition comprising
(a) a protein, a nucleic acid molecule and/or a host cell of the present invention, as defined herein, and
(b) excipient(s) and/or carrier.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to 21” should be interpreted to include not only the explicitly recited values of 1 to 21, but also include individual values and sub-ranges within the indicated range. Thus, included in this numerical range are individual values such as 1, 2, 3, 4, 5 . . . 17, 18, 19, 20, 21 and sub-ranges such as from 2 to 10, 8 to 15, etc. This same principle applies to ranges reciting only one numerical value, such as “at least 80%” or “at least 82%”. Furthermore, such an interpretation should apply regardless of the breadth of the range or the characteristics being described.

### Ageritin and Respective Nucleic Acid Molecules Encoding It

As discussed above, the present invention provides the fungal protein ageritin and paralogs and homologs thereof.

In particular, the present invention provides a protein selected from

a protein comprising an amino acid sequence of SEQ ID NO. 1 or an amino acid sequence of SEQ ID NO. 3, or an amino acid sequence having at least 60% sequence identity, preferably at least 70% sequence identity, more preferably at least 80% or 81% sequence identity to the amino acid sequence of SEQ ID NO. 1 or SEQ ID NO. 3, and/or
a protein encoded by a nucleotide sequence of SEQ ID NO. 2 or SEQ ID NO. 4 or a nucleotide sequence having at least 60% sequence identity, preferably at least 70% sequence identity, more preferably at least 80% or 81% sequence identity to the nucleotide sequence of SEQ ID NO. 2 or SEQ ID NO. 4.

In one embodiment, the present invention provides a protein that consists of the amino acid sequence of SEQ ID NO. 1 or of SEQ ID NO. 3, optionally with a suitable tag, such as a His-tag.

In one embodiment, the present invention provides a protein that comprises an amino acid sequence having at least 60% sequence identity, preferably at least 70% sequence identity, more preferably at least 80% sequence identity, more preferably at least 81% sequence identity, more preferably at least 82% sequence identity, more preferably at least 83% sequence identity, more preferably at least 84% sequence identity, more preferably at least 85% sequence identity, more preferably at least 90%, more preferably at least 92%, more preferably at least 95%, more preferably at least 96% sequence identity, more preferably at least 97% sequence, more preferably at least 98% sequence identity or more preferably 99% sequence identity to the amino acid sequence of SEQ ID NO. 1 or SEQ ID NO. 3.

In one embodiment, the present invention provides a protein that is encoded by a nucleotide sequence that consists of the nucleotide sequence of SEQ ID NO. 2 or SEQ ID NO. 4.

In one embodiment, the present invention provides a protein that is encoded by a nucleotide sequence having at least 60% sequence identity, preferably at least 70% sequence identity, more preferably at least 80% sequence identity, more preferably at least 81% sequence identity, more preferably at least 82% sequence identity, more preferably at least 83% sequence identity, more preferably at least 84% sequence identity, more preferably at least 85% sequence identity, more preferably at least 90%, more preferably at least 92%, more preferably at least 95%, more preferably at least 96% sequence identity, more preferably at least 97% sequence, more preferably at least 98% sequence identity or more preferably 99% sequence identity to the nucleotide sequence of SEQ ID NO. 2 or SEQ ID NO. 4.

Ageritin (wt) amino acid sequence
(verified protein sequence encoded by gene AaeAGT1,
gene ID AAE3_01767):
SEQ ID NO. 1
MSESSTFTTAVVPEGEGVAPMAETVQYYNSYSDASIASCAFVDSGKDKIDKTKLVTYTSRLA
ASPAYQKVVGVGLKTAAGSIVPYVRLDMDNTGKGIHFNATKLSDSSAKLAAVLKTTVSMTEA
QRTQLYMEYIKGIENRSAQFIWDWWRTGKAPA
Ageritin (wt) nucleotide sequence
(verified cDNA sequence of gene AaeAGT1, gene ID AAE3_01767)
SEQ ID NO. 2
atgtccgagtcctctaccttcaccactgcggtagtacctgaaggcgaaggagttgctccaat
ggcagagaccgtgcagtattacaactcctactctgacgcatccatcgcgtcttgcgcatttg
tagactcggggaaggacaaaattgataagaccaagttggtcacgtacaccagccgcctcgcc
gcaagccccgcatatcagaaggtcgtcggcgtcggcctcaaaacggccgcgggctccatcgt
gccctacgtccggctcgacatggacaacaccggcaagggcatccatttcaacgcgactaaac
tctccgacagttccgccaagctcgccgcggtgctcaagacgacggtgtccatgaccgaggca
cagcgaactcaactctacatggagtatatcaagggcatcgagaatcggagtgcgcagtttat
ttgggactggtggaggacgggcaaggctccggcgtga

SEQ ID NO. 3 and SEQ ID NO. 4 are shown further below.

In one embodiment, the protein of the present invention comprises an amino acid substitution in position Y57, D91 and/or K110.

Preferably, the protein of the present invention does not comprise an amino acid substitution in position R87, D89 and/or H98.

As discussed above, the present invention provides nucleic acid molecules.

In particular, the present invention provides a nucleic acid molecule, comprising a nucleotide sequence of SEQ ID NO. 2 or SEQ ID NO. 4, or a nucleotide sequence having at least 60% sequence identity, preferably at least 70% sequence identity, more preferably at least 80% sequence identity, more preferably at least 81% sequence identity, more preferably at least 82% sequence identity, more preferably at least 83% sequence identity, more preferably at least 84% sequence identity, more preferably at least 85% sequence identity, more preferably at least 90%, more preferably at least 92%, more preferably at least 95%, more preferably at least 96% sequence identity, more preferably at least 97% sequence, more preferably at least 98% sequence identity or more preferably 99% sequence identity to the nucleotide sequence of SEQ ID NO. 2 or SEQ ID NO. 4,

preferably coding for a protein according to the present invention.

In one embodiment, the present invention provides a nucleic acid molecule consisting of the nucleotide sequence of SEQ ID NO. 2 or SEQ ID NO. 4.

In one embodiment, the present invention provides a nucleic acid molecule that comprises a nucleotide sequence having at least 60% sequence identity, preferably at least 70% sequence identity, more preferably at least 80% sequence identity, more preferably at least 81% sequence identity, more preferably at least 82% sequence identity, more preferably at least 83% sequence identity, more preferably at least 84% sequence identity, more preferably at least 85% sequence identity, more preferably at least 90%, more preferably at least 92%, more preferably at least 95%, more preferably at least 96% sequence identity, more preferably at least 97% sequence, more preferably at least 98% sequence identity or more preferably 99% sequence identity to the nucleotide sequence of SEQ ID NO. 2 or SEQ ID NO. 4.

In one embodiment, the nucleic acid molecule of the present invention further comprises vector nucleic acid sequences, preferably expression vector sequences.

In one embodiment, the nucleic acid molecule of the present invention further comprises promoter nucleic acid sequences and terminator nucleic acid sequences, and/or comprises other regulatory nucleic acid sequences.

In one embodiment, the nucleic acid molecule of the present invention comprises dsDNA, ssDNA, cDNA, LNA, PNA, CNA, RNA or mRNA or combinations thereof.

### Host Cells, Plants and Fungi Expressing Ageritin

As discussed above, the present invention provides host cells.

In particular, the present invention provides a host cell which contains a nucleic acid molecule according to the present invention and preferably expresses said nucleic acid molecule, and/or expresses the protein according to the present invention.

The Host Cell is Preferably Selected from a Bacterial Cell, a Plant Cell or a Fungal Cell

In one embodiment, the host cell is a bacterial cell:

preferably an Escherichia cell,
such as Escherichia coli
e.g. E. coli K12, DH5alpha, JM101, JM109, BL21, SURE,

In one embodiment, the host cell is a plant cell,

preferably from an agricultural crop and/or an ornamental plant,
such as Zea mays (field corn: dent corn, flint corn, flour corn and blue corn; sweet corn: Zea mays convar. saccharata var. rugosa; popcorn: Zea mays everta), Gossypium spp. (cotton species), Capsicum spp., Solanum tuberosum, Solanum lycopersicum, Nicotiana tabacum, Phaseolus lunatus, Pisum sativum var. macrocarpon, Glycine max, Arachis hypogaea, Triticum aestivum, Avena sativa, Hordeum vulgare, Secale cereale, Malus domestica, Pyrus communis, Prunus spp., e.g. P. avium, P. persica, and P. domestica, Ribes spp., e.g. R. uva-crispa, R. nigrum, and R. rubrum, Vitis vinifera.

In one embodiment, the host cell is a fungal cell,

preferably
from an edible, medicinal or ornamental (cultivated) mushroom,
   such as Agaricus bisporus, Pleurotus ostreatus, P. eryngii, Lentinula edodes, Hericium spp., Volvariella volvacea, Grifola frondosa, Ganoderma spp., Trametes spp, Auricularia polytricha, Flammulina velutipes, Lentinus (Pleurotus) sajor-caju, Hypsizygus tessellatus (Buna-shimeji, Brown Beech Mushroom, and Bunapi-shimeji, White Beech Mushroom)
a yeast cell, preferably a basidiomycete yeast cell,
   such as Ustilago spp., e.g. U. maydis, Microbotryum spp., e.g. M. lychnidis-dioicae, and M. violaceum, Xanthophyllomyces dendrorhous, Rhodotorula spp., e.g. R. glutinis, and R. mucilaginosa, Sporobolomyces (Sporidiobolus) spp., e.g. S. roseus, Mrakia spp., e.g. M. frigida, M. psychrophila, and M. gelida,
from an entomopathogenic or mite-pathogenic fungus,
   such as Beauveria (Cordyceps) bassiana, Hirsutella thompsonii, Isaria (Paecilomyces) spp., e.g. I. fumosorosea, Lecanicillium spp., e.g. L. longisporum, and L. muscarium, Metarhizium spp., e.g. M. anisopliae, and M. acridum, Nomuraea spp., e.g. N. rileyi.

The fungal cell according to the present invention is not a cell from A. aegerita, the fungus where ageritin and its paralog are derived from.

The present invention provides a recombinant protein which is obtained from a host cell of the present invention, wherein the host cell is preferably a cell from Escherichia coli, Zea mays, Gossypium spp. (cotton species), Capsicum spp., Solanum tuberosum, Solanum lycopersicum, Nicotiana tabacum, Phaseolus lunatus, Pisum sativum var. macrocarpon, Glycine max, Arachis hypogaea, Triticum aestivum, Avena sativa, Hordeum vulgare, Secale cereale, Malus domestica, Pyrus communis, Prunus spp., e.g. P. avium, P. persica, and P. domestica, Ribes spp., e.g. R. uva-crispa, R. nigrum, and R. rubrum, Vitis vinifera,

more preferably Escherichia coli, Zea mays, Gossypium spp. (cotton species), Capsicum spp., Solanum tuberosum, Solanum lycopersicum, Nicotiana tabacum, Phaseolus lunatus, Pisum sativum var. macrocarpon, Glycine max, Arachis hypogaea, Triticum aestivum, Avena sativa, Hordeum vulgare, Secale cereale, Malus domestica, Pyrus communis, Prunus avium, Prunus persica, Prunus domestica, Ribes uva-crispa, Ribes nigrum, Ribes rubrum, Vitis vinifera.

In one embodiment, the ageritin of the present invention is recombinantly produced in the cytoplasm of the host cell, such as E. coli. Such recombinant ageritin lacks posttranslational modifications (glycosylations). Furthermore, it preferably shows a higher thermal and chemical stability and a high mutational adaptability.

As discussed above, the present invention provides plants.

In particular, the present invention provides a plant, which
contains a nucleic acid molecule according to the present invention and preferably expresses said nucleic acid molecule,
and/or expresses the protein according to the present invention,
and/or comprises host cell(s) of the present invention,

Preferably, the plant is an agricultural crop and/or an ornamental plant,

such as Zea mays (field corn: dent corn, flint corn, flour corn and blue corn; sweet corn: Zea mays convar. saccharata var. rugosa; popcorn: Zea mays everta), Gossypium spp. (cotton species), Capsicum spp., Solanum tuberosum, Solanum lycopersicum, Nicotiana tabacum, Phaseolus lunatus, Pisum sativum var. macrocarpon, Glycine max, Arachis hypogaea, Triticum aestivum, Avena sativa, Hordeum vulgare, Secale cereale, Malus domestica, Pyrus communis, Prunus spp., e.g. P. avium, P. persica, and P. domestica, Ribes spp., e.g. R. uva-crispa, R. nigrum, and R. rubrum, Vitis vinifera.

Preferably, the plant of the present invention is protected against insect pests.

For example, a Zea mays plant of the present invention is protected against the European corn borer (Ostrinia nubilalis).

In one embodiment, the plant furthermore comprises Bacillus thuringiensis, or Bacillus thuringiensis subspecies israelensis (Bti).

As discussed above, the present invention provides fungi.

In particular, the present invention provides a fungus, which
contains a nucleic acid molecule according to the present invention and preferably expresses said nucleic acid molecule,
and/or expresses the protein according to the present invention,
and/or comprises host cell(s) of the present invention,

Preferably, the fungus is an edible, medicinal or ornamental (cultivated) mushroom, such as Agaricus bisporus, Pleurotus ostreatus, P. eryngii, Lentinula edodes, Hericium spp., Volvariella volvacea, Grifola frondosa, Ganoderma spp., Trametes spp., Auricularia polytricha, Flammulina velutipes, Lentinus (Pleurotus) sajor-caju, Hypsizygus tessellatus (Buna-shimeji, Brown Beech Mushroom, and Bunapi-shimeji, White Beech Mushroom).

Preferably, the fungus of the present invention is protected against insect pests, such as fungus gnats and fungus pests.

For example, fungus gnats (Sciaridae and Phoridae), such as Lycoriella ingenua, are black flies which damage the fruiting bodies grown in mushroom farms, e.g. of Agaricus bisporus.

Further examples of fungus pests are:
Sciaridae/Phoridae feeding on Volvariella volvacea (Indian J Microbiol (April-June 2011) 51(2):200-205)
“Flies” feeding on Pleurotus ostreatus (Rev Argent Microbiol. 2018 April-June; 50(2):216-226)
Camptomyia corticalis and C. heterobia feeding on Lentinula edodes (Mol Ecol Resour. 2013 March; 13(2):200-9.)
Brennandania lambi feeding on Agaricus bisporus (Exp Appl Acarol. 2001; 25(3):187-202.)
Tyrophagus putrescentiae (storage mite) feeding on Agaricus bisporus, Pleurotus ostreatus, Auricularia polytricha and Flammulina velutipes (Environ Entomol. 2015 April; 44(2):392-9.)

In one preferred embodiment, the fungus is an entomopathogenic or mite-pathogenic fungus, such as such as Beauveria (Cordyceps) bassiana, Hirsutella thompsonii, Isaria (Paecilomyces) spp., e.g. I. fumosorosea, Lecanicillium spp., e.g. L. longisporum, and L. muscarium, Metarhizium spp., e.g. M. anisopliae, and M. acridum, Nomuraea spp., e.g. N. rileyi.

e.g. Beauveria bassiana, Isaria fumosorosea. Nomuraea rileyi, Hirsutella thompsonii, Metarhizium anisopliae, Lecanicillium longisporum.

The fungus according to the present invention is not A. aegerita, i.e. the fungus where ageritin and its paralog are derived from.

### Uses as (Bio)Insecticides

As discussed above, the present invention provides the use of
the protein of the present invention, as defined herein,
the nucleic acid molecule of the present invention, as defined herein,
the host cell of the present invention as defined herein, and/or
the plant or the fungus of the present invention, as defined herein,
as bioinsecticide.

The term “bioinsecticide” as used herein refers to biological insecticides, which are generated by organisms, such as plants or fungi, which express insecticidal compounds or components, such as insecticidal proteins, metabolites.

In a preferred embodiment, the use is directed against mosquitoes, preferably disease vector mosquitoes, such as of the viral diseases yellow fever, chikungunya, dengue, zika, West Nile fever, parasitic diseases such as lymphatic filariasis and malaria),

more preferably
Aedes spp. (Aedes aegypti, Ae. japonicus, Ae. albopictus),
Culex spp. and Culex pipiens spp. (C. nigripalpus, C. pipiens, C. tarsalis, C. annulirostris, C. univittatus, C. quinquefasciatus),
Anopheles spp. (Anopheles quadrimaculatus, Anopheles gambiae, Anopheles stephensi, Anopheles maculipennis, Anopheles funestus, Anopheles arabiensis),
Stegomyia (Aedes) aegypti,
Stegomyia (Aedes) albopicta,
Aedimorphus (Aedes) vexans.

More preferably, the use is directed against the larvae of said mosquitoes (“larviciding”).

In said embodiment, the use also contributes to the increase of live quality in the environment of water bodies or inshore waters, since said water bodies or inshore waters will no longer be the breeding ground of mosquitoes.

Toxicity assays revealed a strong larvicidal activity of ageritin, as shown herein, the cytoplasmically synthesized ribotoxin from the mushroom Agrocybe aegerita, against mosquito larvae making this protein an excellent novel biopesticide/bioinsecticide.

Tropical mosquito species like the yellow fever mosquito Aedes aegypti are most important disease vectors for the most important infectious diseases globally, including the severe arboviral diseases such as yellow fever, chikungunya, dengue or zika. Stopping the development from larvae into adult mosquitos by larviciding (killing of their larvae) efficiently can abolish the establishment and spreading of stable populations by such mosquitos and can hence minimize the risk of disease transmission.

Larviciding is currently done by introducing the biopesticide/bioinsecticide Bacillus thuringiensis israelensis (Bti) into bodies of standing water reservoirs as potential sites of egg laying and larval hatching. However, this practice is potentially at risk of becoming ineffective as different genetic reports on Bti resistance development by Ae. aegypti and other mosquitos like Culex quinquefasciatus have been published (see e.g. Cadavid-Restrepo et al., 2012; Suter et al., 2017). Ageritin is therefore a vital alternative to Bti not least as a reserve larvicidal in cases where Bti becomes ineffective. In addition, Bti containing products on the market are known for getting less available to the larvae over time, e.g. due to sinking down the water column or due to being uptaken by other organisms for which they are not toxic.

A prerequisite for the use of ageritin or of its paralog(s) as bioinsecticide is, besides its potency against target insects, its lack of side effects i.e. toxicity towards other cells or organisms. Thus far, we can say that the protein is toxic for insects such as Ae. aegypti, CNS tumor cell lines but not for nematodes including Caenorhabditis elegans. In addition, the mushroom A. aegerita where ageritin is derived from, is a commercially cultivated edible mushroom.

Larviciding for mosquito control is conventionally done on a global scale by Bacillus thuringensis israelensis (Bti) treatment, not least in Switzerland. However, this conventional practice is potentially at risk of becoming ineffective as cases of resistance development to Bti are reported in pestiferous mosquito species such as Ae. aegypti (see e.g. Cadavid-Restrepo et al., 2012). In cases where Bti becomes ineffective due to a potential establishment of Bti resistant Ae. aegypti populations, ageritin is a valuable and highly effective alternative.

In addition, other important disease vector mosquitos on the rise like the common malaria mosquito Anopheles quadrimaculatus in the United States of America, are naturally more difficult to control with Bti. For example, Bti applied as a liquid is heavy and sinks into the water column which makes it less available to surface feeding larvae like the ones of An. quadrimaculatus (Weeks et al., 2018). Ageritin is a small highly soluble protein which should not be so prone to sink into the water column and should thus be more available also to surface feeding larvae like to ones of An. quadrimaculatus.

Moreover, Bti toxicity against for example Ae. aegypti is mediated by synergism effects between the different kinds of crystal proteins (Bti toxins) which are produced during sporulation in Bti. The insecticidal activity of the isolated Bti toxins is magnitudes of order less toxic than the crystal inclusion containing all toxins (Cantón et al., 2011). In contrast to that, ageritin is highly toxic to Ae. aegypti on its own.

Applications of other fungal ribotoxins as biopesticides have been suggested (Olombrada et al., Toxins 2017). In contrast to these secreted ribotoxins, however, ageritin is the first and so far only intracellular ribotoxin produced in the cytoplasm. This feature makes it much easier to produce this toxin recombinantly such as in E. coli and to keep the toxin associated with the bacterial cells if desired for the specific application.

In one embodiment, the use is in combination with further insecticide(s) against mosquito larvae, such as
methionine
temephos
Bacillus thuringiensis subspecies israelensis toxins (Bti toxins),
Lysinibacillus sphaericus powder SPH88,
chitin synthesis inhibitor(s), e.g. diflubenzuron, novaluron,
pyriproxyfen,
methoprene,
anethole,
cinnamaldehyde,
cinnamyl acetate,
or combinations thereof.

Examples of (bio)pesticides against mosquito larvae:

Bacillus thuringiensis subspecies israelensis toxins (Bti toxins) are a mix of crystal (Cry) proteins expressed during B. thuringiensis subsp. israelensis sporulation. Spores mainly contain: Cry11Aa, Cry4Aa, Cry4Ba and Cyt1Aa (Berry et al., 2002).

Lysinibacillus sphaericus powder SPH88 (Pasteur Institute, Paris, France, serotype H5a5b strain 2362; see Suter et al., 2017).

Vectomax CG® (Valent Biosciences Corporation, Libertyville, Ill., USA) is a commercial product available as water-soluble pouches containing a granular formulation that combines 4.5% Bti (serotype H-14, strain AM65-52) and 2.7% L. sphaericus (2362, serotype H5a5b, strain ABTS 1743) spores and insecticidal crystals as active ingredients (see Suter et al., 2017).

Methionine, see Weeks et al. (2018).

Temephos, see Suter et al. (2017).

Pyriproxyfen, see Suter et al. (2017).

Diflubenzuron and novaluron, both are chitin synthesis inhibitors. Diflubenzuron can be used together with Bti, see Suter et al. (2017).

Anethole shows larvicidal activity against Aedes aegypti, see Cheng et al. (2004). Cinnamaldehyde shows larvicidal activity against Aedes aegypti larvae, see Cheng et al. (2004). Cinnamyl acetate shows larvicidal activity against Aedes albopictus larvae, see Cheng et al. (2009).

In a preferred embodiment, the use is directed against insect pests attacking crop plants and/or ornamental plants,

such as

### Lepidoptera,

such as Ostrinia nubilalis (European corn borer), Spodoptera exigua, S. frugiperda, S. littoralis, Pectinophora gossypiella, Heliothis virescens, Helicovera zea, Chrysodeixis includes, Helicoverpa armigera, cutworms (various species including Agrotis spp., Peridroma saucia, Nephelodes minians), hornworms (Manduca quinquemaculata, Manduca sexta), Lepidoptera complex (e.g. rice stem borers Chilo auricilius, C. suppressalis, Scirpophaga incertulas, S. innotata), Grapholita funebrana, Phthorimaea operculella, Elasmopalpus lignosellus),

### Coleoptera,

such as Colorado potato beetle (Leptinotarsa decemlineata), Diabrotica spp., Anthonomus grandis (boll weevil), Oulema melanopus, O. gallaeciana
   sucking insects
such as Thrips (Thysanoptera, e.g. Thrips tabaci, Frankliniella schultzei), Aphids (Hemiptera, e.g. Acyrthosiphon pisum, Aphis gossypii, Dysdercus koenigii, Creontiades dilutes), white flies (Homoptera),

### Arachnida

such as Tetranychus urticae, Panonychus ulmi (European red mite).

In one embodiment, said use is in combination with further insecticide(s), such as
Bacillus thuringiensis toxins (Bt toxins),
azadirachtin,
chitin synthesis inhibitor(s), e.g. diflubenzuron,
tebufenozide,
methoprene,
malathion,
or combinations thereof.

Examples of (bio)pesticides against insect pests attacking crop plants:

Bacillus thuringiensis toxins (Bt toxins) are mediated (mainly) by different crystal proteins expressed by B. thuringiensis. Approved Bt genes include single and stacked (event names bracketed) configurations of:
Cry1A.105 (MON89034)
CryIAb (MON810)
CryIF (1507)
Cry2Ab (MON89034)
Cry3Bb 1 (MON863 and MON88017)
Cry34Ab1 (59122)
Cry35Ab1 (59122)
mCry3A (MIR604)
Vip3A (MIR162).

Azadirachtin is used against Bt toxin resistant pests, such as the African cotton leafworm Spodoptera littarolis.

Diflubenzuron is used for killing fungus gnat larvae in greenhouse soil.

Tebufenozide is used to control caterpillar pests.

In one embodiment, the use is
against mite (Acaridae),
such as against Brennandania iambi, Tyrophagus putrescentiae (storage mite), Panonychus ulmi (European red mite), Tetranychus urticae,
against fungus gnats and fungus pests,
such as against Sciaridae and Phoridae (such as Lycoriella ingenua), Camptomyia corticalis, C. heterobia, “flies”,
against storage pests,
such as Bruchids [Callosobruchus chinensis (L.) and Callosobruchus maculatus (F.)], Caryedon serratus, storage insect pests, including the Indian meal moth, Plodia interpunctella (Hübner) (Lepidoptera: Pyralidae); Sitophilus spp. (Coleoptera: Curculionidae); and their natural enemies [e.g., Cephalonomia tarsalis (Ashmead) (Hymenoptera: Bethylidae), and Anisopteromalus calandrae (Howard) (Hymenoptera: Pteromalidae)],
in hygiene,
such as against cockroaches, ants, termites and bed bugs,
   and/or
in crop protection,
such as against slugs, blossom beetle, rape stem beetle, Aphids, Drosophila suzukii, raspberry weevil, caterpillars, fungus gnats, European grape vine moth, stem borers, European red mite (Panonychus ulmi),
   wherein the use can be in combination with further insecticide(s), such as insecticides disclosed herein.

For example, the use against mites can be in combination with Permethrin.

### Bioinsecticide Compositions

As discussed above, the present invention provides a (bio)insecticide composition.

Said composition comprises
(a) a protein of the present invention, a nucleic acid molecule of the present invention and/or a host cell of the present invention, and
(b) excipient(s) and/or carrier.

In one embodiment, the composition furthermore comprises:
(c) further active agent(s),
such as insecticide(s) or pesticide(s).

Examples for further active agent(s) are (bio)pesticides against mosquito larvae and/or (bio)pesticides against insect pests attacking crop plants, as described above, such as:
methionine
temephos
Bacillus thuringiensis subspecies israelensis toxins (Bti toxins),
Lysinibacillus sphaericus powder SPH88,
chitin synthesis inhibitors, e.g. diflubenzuron, novaluron,
pyriproxyfen,
methoprene,
anethole,
cinnamaldehyde,
cinnamyl acetate,
Bacillus thuringiensis toxins (Bt toxins),
azadirachtin,
tebufenozide,
malathion,
or combinations thereof.

In one embodiment, the composition is formulated as
a solution, such as liquids e.g. liquid concentrates,
a powder (such as a wettable powder),
granules,
baits.

Liquid concentrates and powders can be used as spray.

The formulations can further comprise spreading and/or sticking agent(s).

### Preferred Embodiments

In the present study, we identified the ageritin-encoding gene AaeAGT1 and the neighboring gene AaeAGT2 on the chromosomal A. aegerita DNA encoding a paralogous protein, from the recently published genome sequence of A. aegerita (Gupta et al., 2018). Transcriptional profiling of AaeAGT1 revealed a very strong induction of AaeAGT1 expression during mushroom formation with a trend towards specificity to the developing mushroom cap. The predicted amino acid sequence of ageritin revealed marked differences to all other fungal ribotoxins described so far. Employing heterologous expression of ageritin or its paralog(s) in E. coli, we subsequently checked for insect and nematode toxicity as well as for in vitro ribonucleolytic activity of wild type and mutagenized ageritin. Finally, we functionally characterized the AaeAGT2-encoded ageritin paralog, which we identified based on its high sequence similarity to AaeAgt1. Searching for homologies of sequences we found that homologs of ageritin are widespread among a variety of fungal species including several plant pathogenic fungi.

### Abstract

Here, we report on a ribotoxin from Agrocybe aegerita referred to as ageritin and its paralog, the first ribotoxin described from Basidiomycota. The amino acid sequence markedly differs from Ascomycota ribotoxins. It does not contain any signal peptide for classical secretion, which represents the first cytoplasmic ribotoxin. The ageritin-encoding gene AaeAGT1 is highly induced during fruiting. AaeAGT1-cDNA was cloned and expressed in E. coli for further study. Toxicity assays showed a strong insecticidal activity against Aedes aegypti whereas no toxicity was found against nematodes. The rRNase activity of the ageritin was confirmed in vitro using lysate ribosomes. Mutagenesis studies showed correlation between in vivo and in vitro activities indicating that the entomotoxicity is mediated by the ribonucleolytic cleavage. The strong toxicity of ageritin against mosquito larvae makes it a new biopesticide.

### Results

### 1. Identification of the Ageritin Encoding Gene from the A. aegerita Genome Sequence

By BLASTing the published 25 N-terminal residues of ageritin (Landi et al., 2017) against the predicted proteome of A. aegerita, we were able to identify the ageritin-encoding gene from the fungus. It will be referred to as AaeAGT1 (gene ID AAE3_01767). In addition, we found a neighboring gene on the chromosomal DNA of A. aegerita encoding a paralogous protein, which is referred as AaeAGT2 (gene ID AAE3_01768). Compared to ageritin, the deduced amino acid sequence of AaeAgt2 displays 63% sequence identity (FIG. 1A). Interestingly, the predicted amino acid sequence of AaeAgt1 neither shows sequence similarity to any of the known ascomycete ribotoxins nor does it possess a known signal sequence for classical secretion, suggesting a novel type of ribotoxin.

SEQ ID NO. 1 shows the ageritin (wt) amino acid sequence, the verified protein sequence encoded by gene AaeAGT1, gene ID AAE3_01767.

SEQ ID NO. 2 shows the ageritin (wt) nucleotide sequence, the verified cDNA sequence of gene AaeAGT1, gene ID AAE3_01767.

SEQ ID NO. 3 shows the amino acid sequence of the paralogous
protein encoded by the gene AaeAGT2:
MSDPSAPGLEEGTEISPMAETVQTFASYSEASVAACKWVNSGKTQIDPAQLILYKNTLPASP
AYGKIVGVGLKFTAEVDFCRLDMDNTGKGIHFNAKQRDDQSKKLAAVIKPTVALSEAQRTQL
YMEYIKGLENRSAQFIWEWWSTGKAPA
SEQ ID NO. 4 shows the nucleotide sequence of the gene AaeAGT2
atgtctgacccatccgcacccggactcgaagagggcaccgaaatctcacccatggccgagac
ggtgcagaccttcgcgagctactccgaggcctccgttgccgcttgcaagtgggtcaactcgg
gaaagacccagattgacccagcccagctcatcctgtacaagaacaccctccccgcaagcccc
gcatacggcaaaatcgtcggcgtcggcctaaaattcaccgccgaggtcgacttctgccgact
cgacatggacaacacaggcaagggcatccacttcaacgccaagcagcgggacgaccagtcca
agaagctcgcggcggtgatcaagccgactgttgcgttgagcgaggcccagcgcacgcagctg
tacatggagtatatcaaggggctcgagaacaggagtgcgcagtttatttgggagtggtggag
caccgggaaggcaccggcgtga

### 2. Expression of Reference Genes During A. aegerita Vegetative Growth and Mushroom Formation

Three genes with a high expression stability during vegetative mycelium growth as well as throughout the fruiting body development have been identified within the genome sequence of this dikaryotic strain (Gupta et al., 2018):
AaeIMP1 (gene ID AAE3_02268),
AaeTIF1 (gene ID AAE3_07769) and
AaeARP1 (gene ID AAE3_11594).

Blast search for the deduced amino acid sequences against the UniProt database (www.uniprot.org) revealed AAE3_02268 encoding a putative importin, AAE3_07669 coding for a putative translation initiation factor and AAE3_11594 encoding a putative autophagy related protein. The manually designed primer pairs for each putative reference gene showed efficiencies of 104%, 109% and 107% with a regression coefficient of 0.998, 0.996 and 0.998 for AaeIMP1, AaeTIF1 and AaeARP1, respectively. Validation of the reference genes according to their Cq-values for 12 analyzed samples (FIG. 6) with the NormFinder algorithm revealed values of 0.119, 0.126 and 0.167 for genes AaeTIF1, AaeIMP1 and AaeARP1, respectively. geNorm showed a similar trend with values of 0.29 for AaeTIF1 and AaeIMP1 as well as 0.33 for AaeARP1. Overall, on the basis of the NormFinder and geNorm validation, the reference genes AaeTIF1 (gene ID AAE3_07769) and AaeIMP1 (gene ID AAE3_02268) are the best combination for qRT-PCR based transcription analyses of A. aegerita genes expressed during vegetative mycelium growth or fruiting body development.

### 3. Expression of the Ageritin Gene During Fruiting Body Formation of A. aegerita

In order to assess the expression of both the ageritin encoding gene AaeAGT1 and the paralogous gene AaeAGT2, quantitative real-time reverse transcription PCR (qRT-PCR) analyses were carried out using RNA from different dikaryotic developmental stages of A. aegerita AAE-3. Using vegetative mycelium as a reference, a strong upregulation of AaeAGT1 expression was found during the whole fruiting body development of A. aegerita AAE-3. Upregulation was not found in the case of AaeAGT2 (FIG. 1B).

The results are shown in FIG. 1B as a relative expression compared to the expression by the vegetative mycelium (I). AaeAGT1 expression becomes highly upregulated by a factor of 87 during the shift into the ready-to-fruit mycelium (stage II). The expression level peaks with a factor of 160 when the first macroscopically visible complex multicellular plectenchymatic structures of fruiting body development can be spotted, which are referred to as fruiting body (FB) initials (stage III). Although a less pronounced expression increase was both recorded in subsequently emerging FB primordia (stage IV, AaeAGT1 expression upregulated by a factor of 19) and in the stipe plectenchyme of the thereafter developing young FBs (stage Vs, AaeAGT1 expression upregulated by a factor of 5), an increased expression was again observed within the cap plectenchyme of young FBs (stage Vc, 100-fold).

AaeAGT2 showed no clear change in expression of the magnitudes of the AaeAGT1 amplicon varying from 0.4 in stage IV to 2.3 in stage III. Both gene's Cq values in the calibrator stage were very similar with an average 25.55 for the AaeAGT1 amplicon and an average 25.73 for its paralog AaeAGT2 indicating comparable template amounts for a ‘1-fold’ expression for the two genes in this cDNA pool.

### 4. Heterologous Expression and Purification of Ageritin from E. coli Cells

In contrast to all other ribotoxins, AaeAgt1 does not contain a signal peptide for a classical secretion and is thus predicted to be localized in the cytoplasm. Based on the lack of a signal peptide in the predicted amino acid sequence of ageritin, we expressed the protein in the cytoplasm of E. coli. It was shown that both, untagged and N-terminally His₈-tagged ageritin were expressed in a fully soluble form in the bacterial cytoplasm (FIG. 7A). The solubility of the His₈-tagged ageritin allowed us to purify the recombinant protein for the in vitro experiments (FIG. 7A). His₈-ageritin was purified over Ni-NTA affinity columns to homogeneity (FIG. 7B). The purified ageritin protein was used for in vitro ribonucleolytic cleavage assay and for toxicity assays with the insect sell line Sf21.

### 5. rRNA Cleavage Activity of Recombinant Ageritin

Ribonucleolytic activity of ageritin was assayed against ribosomes of a rabbit reticulocyte lysate. The results indicate that ageritin, same as α-Sarcin (Chan et al., 1983), acts on the 28S rRNA subunit of ribosomes and releases as classical ribotoxin cleavage product, the α-fragment (FIG. 1B). We observed a similar pattern on a gel for the two samples that were either treated with only α-Sarcin or simultaneously with both, α-Sarcin and ageritin, suggesting that the cleavage site for ageritin is the very close by or identical to that of α-Sarcin (FIG. 1B).

### 6. Entomo- and Nematotoxicity of Wild Type and Recombinant Ageritin

The ageritin-expressing E. coli cells and the tagged version were tested for insecticidal activity against L3-larvae of A. aegypti. After four days, significant larval mortality was recorded (FIG. 2A). Concomitantly, feeding inhibition was measured with the change in OD₆₀₀ of a respective bacterial suspension. Larvae feeding on “empty vector” cells reduced the bacterial concentration resulting in a drop of OD₆₀₀. In fact, the bacterial consumption was significantly reduced in the case of Cgl2-, ageritin- or His₈-ageritin-expressing bacteria (FIG. 2B).

The purified ageritin protein also showed toxicity against the insect cells of Spodoptera frugiperda Sf21 in a concentration dependent manner (FIG. 2C).

After confirming the insecticidal activity of the ribotoxin, we tested its activity against five different bacterivorous nematode species (Table 4). Synchronized L1 larvae of the nematodes (Caenorhadbitis elegans, C. briggsae, C. tropicalis, Distolabrellus veechi and Pristionchus pacificus) were fed with bacteria expressing ageritin, and their development was assessed after 2 days. Interestingly, ageritin was not active against any of the tested nematode species, pointing to specificity for certain organisms (FIG. 8).

### 7. Correlation Between rRNA Cleavage and Toxicity

The gene sequence of ageritin was aligned against its top ten homologs. Six conserved residues were chosen for mutation into alanine (FIG. 3A). All the mutants were expressible in a highly soluble manner in the cytoplasm of E. coli (FIG. 7C). To assess the importance of the mutated residues for the insecticidal activity of ageritin, we performed a toxicity assay against Ae. aegypti larvae. No toxicity was measured for three conserved-site mutants (R87A, D89A and H98A) while the other three mutants (Y57A, D91A and K110A) performed similar to the wild type ageritin (FIG. 3B).

In a second approach we measured the change in OD₆₀₀ on a daily basis to check on larval feeding inhibition. The toxic constructs inhibited feeding whereas the reduction in OD₆₀₀ by non-insecticidal bacterial strains was in line with the control (FIG. 3C). In order to differentiate between the effect level of the three point mutations (R87A, D89A and H98A) which had shown significant reduction in ageritin toxicity, the mosquito larvae were allowed to develop into adults. The mutant construct D89A showed complete loss of toxicity. All larvae developed into imagines (FIG. 3D). An average of 40% of the larvae developed into adults when the other two conserved site mutants (R87A and H98A) were assayed. This indicates that partial toxicity in these two mutants is still present (FIG. 3D).

Ageritin single-site mutant constructs were produced in E. coli BL21 in a fully soluble manner and purified over Ni-NTA columns (FIG. 7D). Subsequently, ribonucleolytic activity of the purified proteins were assessed against ribosomes of rabbit reticulocyte lysate. Interestingly, all of the three single-site mutants which were non-toxic in vivo against Ae. aegypti larvae were also inactive in the ribosome cleavage assay (FIG. 3E). The single site mutants that were still toxic in vivo performed similar to the wild type ageritin. This means that they all released the 400 nucleotide long ribotoxin cleavage product, the α-fragment (FIG. 3E). Combined with our insecticidal assay data, the in vitro results demonstrate that the insecticidal toxicity is based on ribonucleolytic activity of the ageritin.

### 8. Functional Comparison Between Ageritin and the Ageritin Paralog

Given the different expression dynamics of AaeAGT2 and AaeAGT1 throughout the developmental stages of the fungus and the recorded bioactivity spectrum of AaeAgt1, we focused on a potential bioactivity of AaeAgt2.

AaeAGT2 was expressed in E. coli BL21 in the same manner as AaeAGT1, and tested against Ae. aegypti and bacterivorous nematodes. The ageritin paralog was also expressed as highly soluble protein in the bacterial cytoplasm (FIG. 4A).

Interestingly, the ageritin paralog was similarly active against Ae. aegypti larvae (FIG. 4B, suggesting the conservation of both the in vitro and in vivo functions between the two paralogous proteins. The weaker entomotoxic activity of the untagged versus a His₈-tagged version can be explained by its significantly lower expression in E. coli (FIG. 4A).

### 9. Distribution of Ageritin Homologs in the Fungal Kingdom

Ageritin homologs were searched using JGI MycoCosm portal (Grigoriev et al., 2014). A high degree of sequence conservation was found among ageritin homologues proteins within different fungal species. The top 30 hits were retrieved from the MycoCosm portal and their phylogenetic relationships were designed as a circular cladogram (FIG. 5). All 30 hits, all originate from basidiomycetes including several plant-pathogenic species such as Rhizoctonia solani, Moniliophthora perniciosa, Rigidoporus microporus and Pleurotus eryngii.

Interestingly, despite ageritin being a cytoplasmic protein, several of its homologs possess known signal sequences, which however resembles more closely the secreted ribotoxins of ascomycetes.

### Discussion

Ribotoxins are suggested to be part of the fungal defense system against insect predators (see e.g. Olombrada et al., 2013).

This is corroborated by the results of the present work, in which we report the full amino acid sequence and the gene encoding a novel type of ribotoxin named ageritin from the edible mushroom A. aegerita. Ageritin is so far the first ribotoxin identified from the phylum Basidiomycota. Its sequence is very different from those of the well-known ribotoxins of Ascomycota. Furthermore, it is the first cytoplasmic ribotoxin discovered up to now.

Our results demonstrate high toxicity of ageritin against Ae. aegypti larvae, suggesting that it plays an important role as a natural defense mechanism of A. aegerita against insects pests of mushrooms such as fungus gnats. In cultivation facilities of the cultivated edible mushroom Agaricus bisporus (white button mushroom), pestiferous fungus gnats like Lycoriella ingenua (Sciaridae) can cause severe damages, e.g. by larval feeding on the button mushroom cultures (Cloonan et al., 2016). Insecticidal activity is a common feature of fungal ribotoxins (Herrero-Galan et al., 2013). Anisopilin and hirsutellin A, two previously described ribotoxins (Yang et al., 1996; Herrero-Galan et al., 2008) had been shown to exert cytotoxicity against insect cell lines. Similarly, ageritin showed cytotoxicity against the insect cell line Spodoptera frugiperda Sf21, albeit at a higher concentration than the well-studied α-Sarcin, suggesting that ageritin is either less active than α-Sarcin or the reaction conditions were not optimal. The in vitro ribonucleolytic assay with ribosomes of rabbit reticulocyte lysate indicates that the latter is likely to be true, since, the cleavage of 28S rRNA was slower for ageritin than α-Sarcin.

Furthermore, we investigated whether the insecticidal activity of ageritin is dependent on its ribonucleolytic activity. The putative catalytic residues of ageritin could not be identified by comparison with the previously characterized ribotoxins due to the lack of sequence homology. However, previous studies had shown that catalytic residues of ribotoxins mostly consist of acidic and basic residues. The charged amino acids of ribotoxins had been found to support binding to the negatively charged rRNA facilitating interactions with target ribosomes (Herrero-Galan et al., 2012). For instance, the active site of α-Sarcin consists of histidine and a glutamic acid (H50, E96 and H137) (Lacadena et al., 1999). Therefore, we mutated the conserved charged amino acid residues (R87, D89, D91, H98 and K110) and tyrosine (Y57) in the ageritin amino acid sequence. Three (R87A, D89A and H98A) of the six mutations abolished both insecticidal activity and in vitro ribonucleolytic cleavage while the other three mutants (Y57A, D91A and K110A) did not affect neither in vivo nor in vitro activity of ageritin. The mutant studies indicate that the insecticidal activity of ageritin depends on the rRNA cleavage activity, and the residues R87, D89 and H98 are part of the catalytic site of ageritin.

While nematodes are important fungal predators in the environment, no activity of ribotoxins against nematodes has been reported so far. As ageritin differs from other ribotoxins in amino acid sequence and structure, we tested the susceptibility of five nematode species (C. elegans, C. briggsae, C. tropicalis, D. veechi and P. pacificus) against ageritin. However, nematotoxicity results show that ageritin is inactive, at least against these five nematode species, suggesting the ribosomes are either resistant or not accessible for ageritin. These findings are in line with previous studies on the activity spectrum of ribotoxins where they exhibit specificity to insect cells due to the special structure and composition of insect cell membrane (Olombrada et al., 2014).

As outlined further above, the entomotoxic activity is conserved between ageritin and its paralog. Ribotoxins cleave the ribosomal RNA in the universally conserved GAGA tetra loop found in the sarcin-ricin loop of the large ribosomal subunit. Therefore, all known ribosomes are potentially susceptible and a ribotoxin producing fungus has to avoid self-intoxication. Protection can be achieved by preventing the active ribotoxin to get in touch with ribosomes by compartmentalization and efficient secretion systems. However, unlike all previously described ribotoxins, ageritin has no known signal peptide sequence and is thus either retained in the cytoplasm or secreted using a yet unknown unconventional secretion pathway in A. aegerita.

We could express ageritin in a highly soluble manner in the cytoplasm of E. coli.

The high toxicity of ageritin against mosquito larvae shows that ageritin and its paralog(s) are highly suitable as new bio-insecticides. Novel mosquito control agents are needed since insecticide resistance to currently available insecticides is a key challenge and more and more insecticides are phased out. Protective interventions such as insecticide-treated bed nets and indoor residual spraying become more and more ineffective. In addition, the increasing spread of invasive Aedes mosquitoes leads to emergence of arboviral diseases such as dengue and chikungunya in more temperate regions (Barzon, 2018). For example, Ae. albopictus has invaded large parts of southern Europe. This insect is an efficient vector viral diseases of dengue, chikungunya and zika virus (Kampen et al., 2017).

### Further Preferred Embodiments

Ageritin, a ribotoxin, is used for fungal self-protection against parasites. Outside the fungal host, ageritin has shown dosage dependent insecticidal activity towards mosquito larvae of the species Aedes aegypti, an important vector of tropical diseases, which is not indigenous in Europe with the exception of Madeira and Georgia. Therefore, a direct close relationship between the fungi and the mosquito can be excluded.

Ageritin, since produced naturally by a fungus, can be considered as a bioinsecticide. New insecticides are urgently needed since regulatory authorities are taking more and more chemical synthetic insecticides from the market due to safety reasons.

### Pest Insects Presenting a Target for Ageritin

### Formulation of an Insecticidal Agent

The mode in which an insecticide is presented to pest insects is crucial. The active substance has to be brought in contact with the site of action within the insect. Most of the chemical insecticides have a major advantage since they pass the epidermis due to their lipophilic properties. Microbial insecticides such as Bacillus thuringiensis have to be ingested by the insects in order to display their insecticidal activity. Ageritin, presented within E. coli to mosquito larvae is also considered as a microbial insecticide. Thus, insecticides, which have to be ingested, need to be formulated and applied in such a way that they are taken up by the target insects in high enough dosages.

A preferred embodiment is the transformation of culture plants with genes expressing insecticides. This technique is presently confined to genes expressing toxins of B. thuringiensis, and used against pest insects, which ravage in the inside of culture crops. Depending on the specificity of ageritin, plant protection can be extended to pest insects feeding on the outside, e.g. on leaves and roots.

### Expression of Ageritin in Culture Plants

For the development of GM-plants (genetically modified), the same approach can be used as in the case of cry-genes (transformation of plants with B. thuringiensis genes expressing insecticidal proteins in plants).

In 2018, the total global acreage of biotech crops amounted to 190 mio acres. Table 1 presents GMO-crops used in the field for the protection against pest insects. They all contain cry-genes of B. thuringiensis.

TABLE 1
Transformation of major culture crops with the ageritin gene for
protection against pest insects.

| | | |
|---|---|---|
| Target insects | Crops | Significance/Remarks |

Lepidoptera such as

| | | |
|---|---|---|
| Ostrinia nubilalis | corn | Bt-corn is world-wide established. |
| (European corn borer) | (maize) | In the US 90% of the corn crop is |
| | | based on Bt-corn. Ageritin-corn |
| | | can be used for: |
| | | live-stock feed, |
| | | sweeteners, such as corn-syrup, |
| | | fuel production, |
| | | industrial uses, |
| Spodoptera exigua, | cotton | Fiber |
| S. frugiperda, | | Animal feed |
| Pectinophora | | Cotton seed-oil |
| gossypiella, | | |
| Heliothis virescens, | | |
| Helicoverpa zea | | |
| Chrysodeixis includes, | soybeans | Cattle feed, vegetable oil, tofu |
| Helicoverpa zea, | | and retail food products. |
| Helicoverpa armigera | | |
| Cutworms, various | brinjal | Important staple vegetable in |
| species, including: | (aubergine) | South-East Asia |
| Agrotis spp. | | |
| Peridroma saucia | | |
| Nephelodes minians | | |
| Hornworms: Manduca | | |
| quinquemaculata, | | |
| Manduca sexta | | |
| Lepidopteran complex | rice | Approved by the Chinese |
| (e.g. rice stem borer) | | Ministry of Agriculture in 2009 |

Coleoptera such as

| | |
|---|---|
| Colorado potato beetle | potatoes, |
| (Leptinotarsa | corn, |
| decemlineata) | soybeans |

Sucking insects such as

| | |
|---|---|
| Thrips (Thysanoptera) | corn, |
| Aphids (Hemiptera) | cotton, |
| white flies (Homoptera) | brinjal |

### Ageritin or its Paralog(s) for use as a Conventional Insecticide in Plant Protection in Different Formulations

Large-scale production of ageritin for field application: It has been shown that ageritin can be produced in cells of E. coli. Ageritin can be produced under controlled conditions in bioreactors, similar to the manufacturing of Bacillus thuringiensis. Following the growth phase, E. coli cells are harvested either by centrifugation and/or by a spray-drying process. The dried E. coli cells retain their insecticidal activity. The concentrated or dried cell material is formulated depending on the designated use as either wettable powders, liquid concentrates, granules or baits.

TABLE 2
Ageritin for use as conventional insecticide in crop protection

| Organism | Crop | Formulation |
|---|---|---|
| Slugs | Arable farming | Baits |
| Seed treatment | Arable farming | |
| Blossom beetle | Canola | Sprays |
| Rape stem weevil | | |
| Aphids | Different crops | Sprays |
| Drosophila suzukii | Soft fruits, grapes | Sprays |
| Raspberry weevil | Raspberries and other fruits and | Baits |
| | plants | |
| Diptera | Many plants, especially vegetables | Sprays |
| Caterpillars | | |
| Fungus gnats | Indoor plants, indoor-farmed | Liquids |
| | edible mushrooms, e.g. Agaricus | |
| | bisporus (button mushroom) | |
| European grape vine | Vine | Liquids |
| moth | | |

### Ageritin or its Paralog(s) as an Insecticide in Public Health and Hygiene

Novel insecticides in the sectors of public health and hygiene are in urgent need. The emphasis in public health lies on the control of mosquitoes representing vectors of human infectious diseases. The main vectors are listed in Table 3a. It has to be added that the approach with ageritin is of special interest since we have clearly demonstrated its mosquitocidal activity.

TABLE 3a
Use of ageritin or its paralog(s) in public health, the control of
mosquito vectors of human diseases

| Public Health | |
|---|---|
| Mosquitoes | Diseases |
| Aedes spp. | |
| Aedes aegypti | Dengue, Chikungunya, Zika |
| | Yellow fever |
| Aedes albopictus | Dengue, Chikungunya, Zika |
| Culex spp. | |
| Culex pipiensis complex | West Nile fever |
| Culex quinquefasciatus | Lymphatic filariasis |
| Anopheles spp. | |
| Anopheles gambiae, An. | Malaria |
| funestus, An. arabiensis | |

TABLE 3b
Use of ageritin or its paralog(s) for the control of insects causing
hygienic problems

| Hygiene | Formulation |
|---|---|
| Cockroaches | Baits |
| Ants, termites | Baits |
| Bed bugs (these insects are of | Beauveria bassiana transformed |
| increasing importance) | with the gene expressing ageritin |

The following examples and drawings illustrate the present invention without, however, limiting the same thereto.

### BRIEF DESCRIPTION OF THE DRAWINGS

FIG. 1. Expression pattern and in vitro rRNA cleavage activity of ageritin.

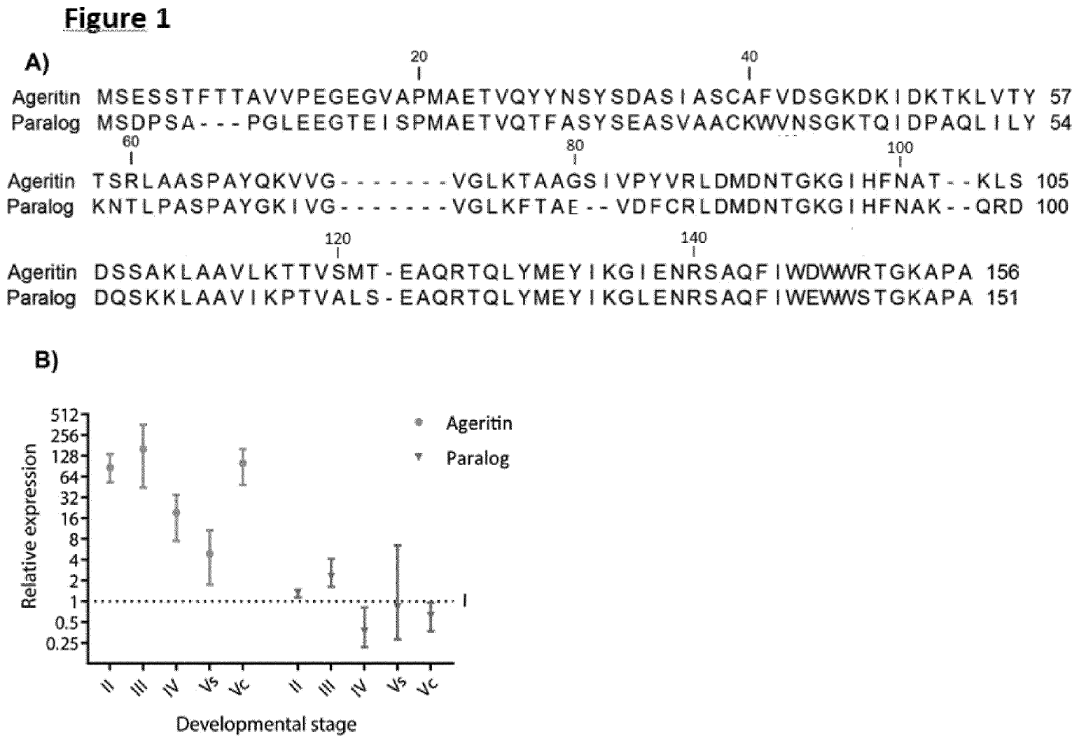

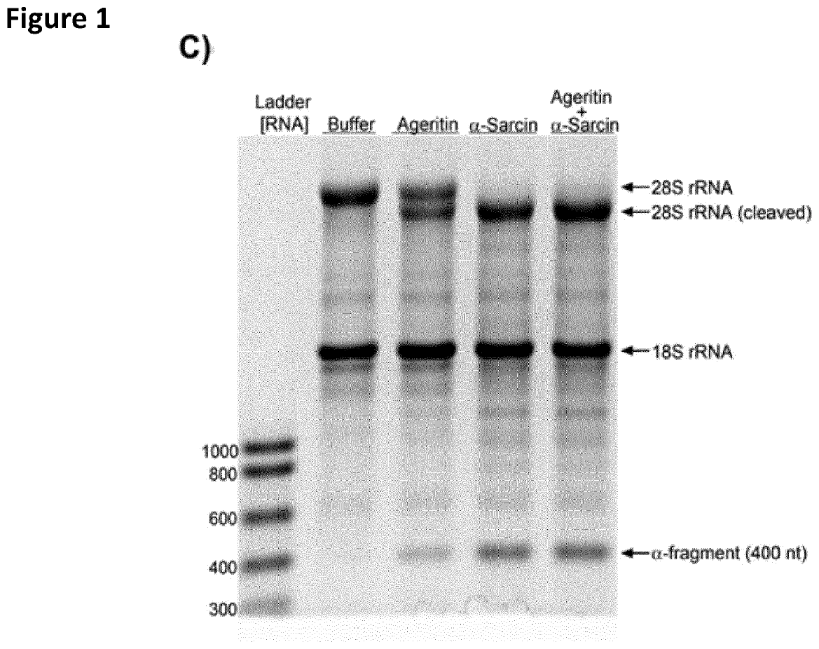

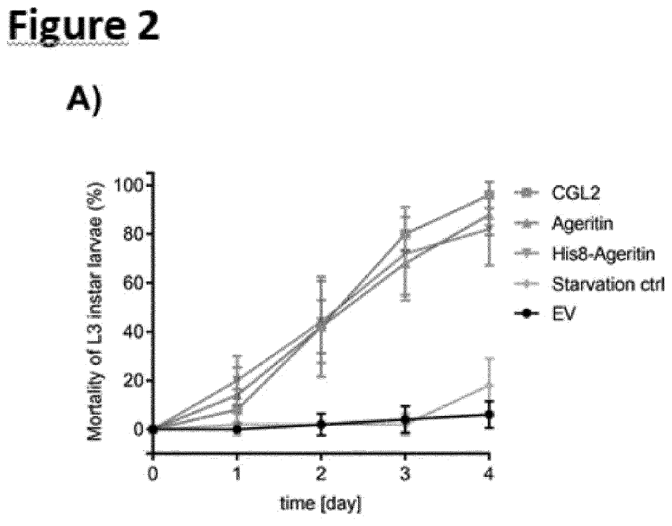

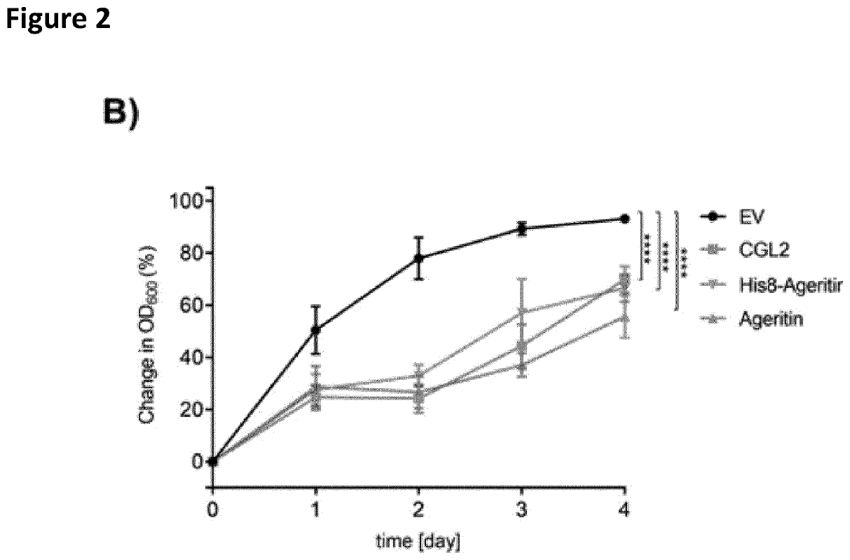

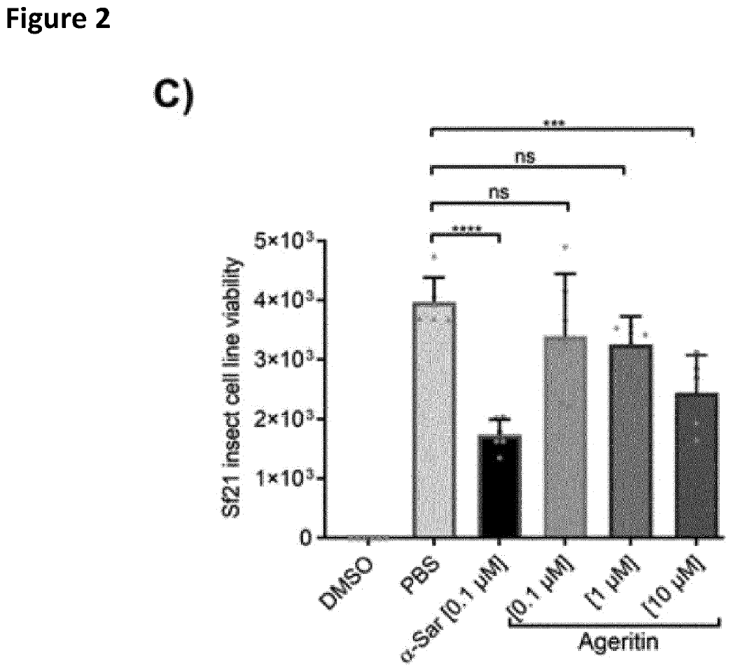

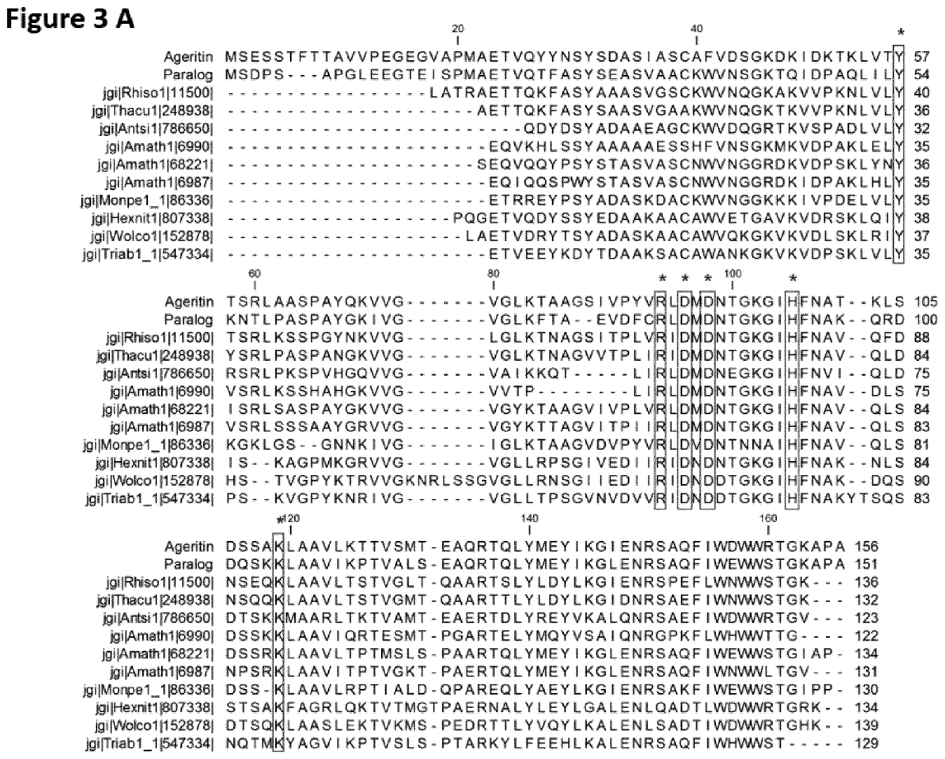

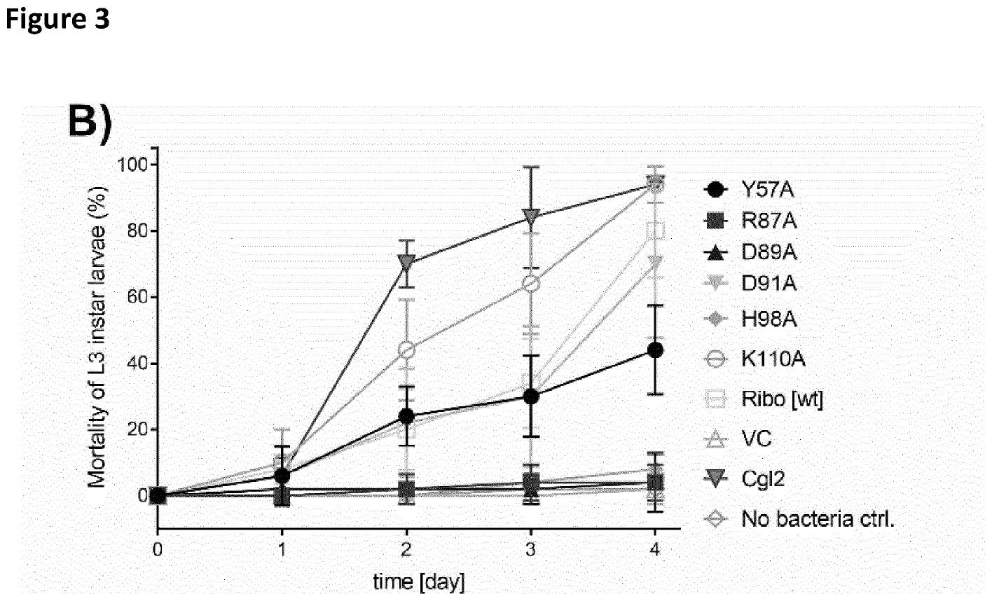

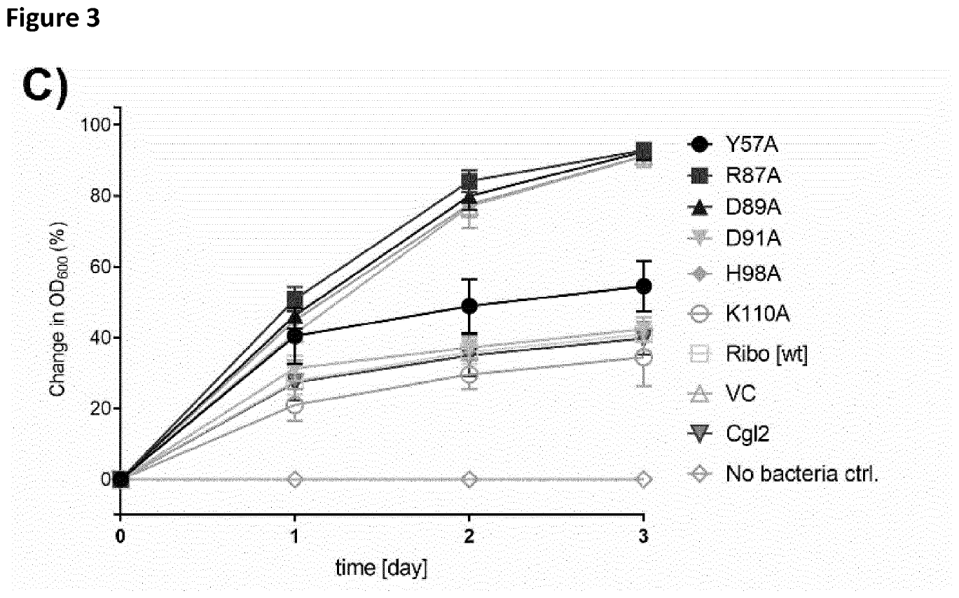

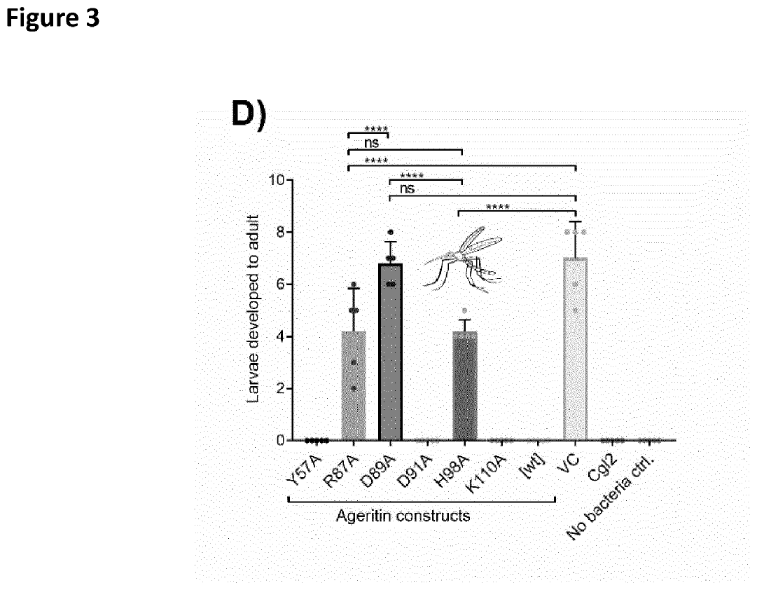

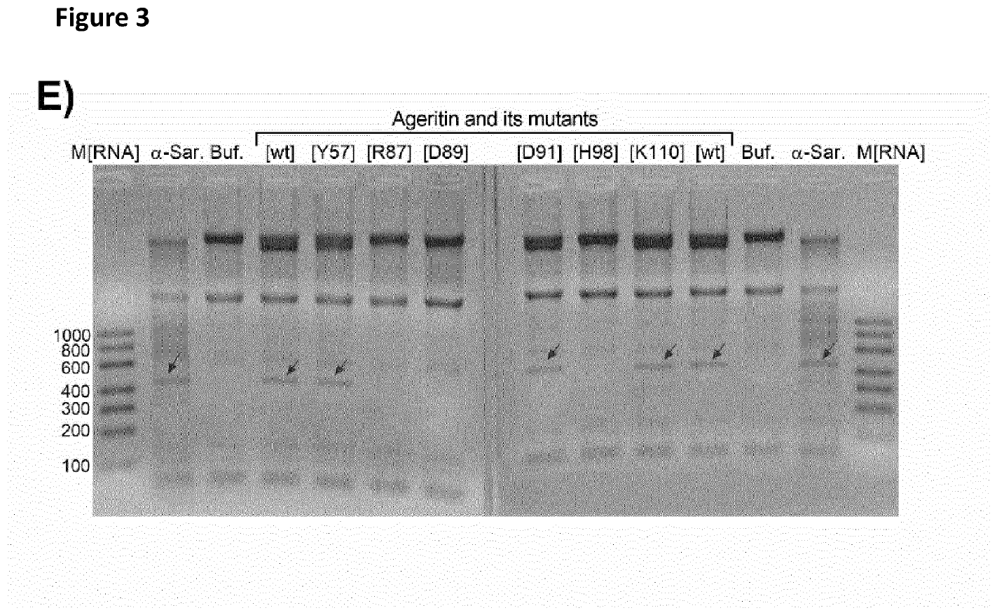

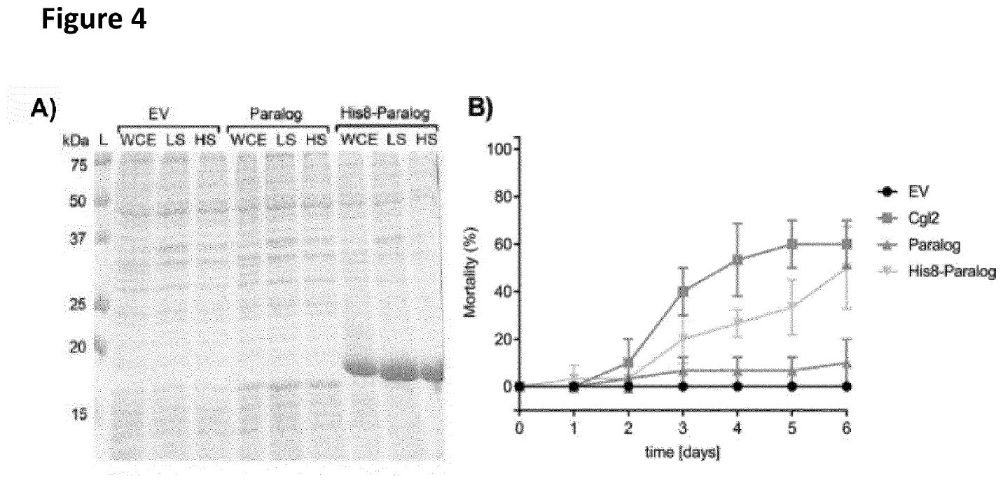

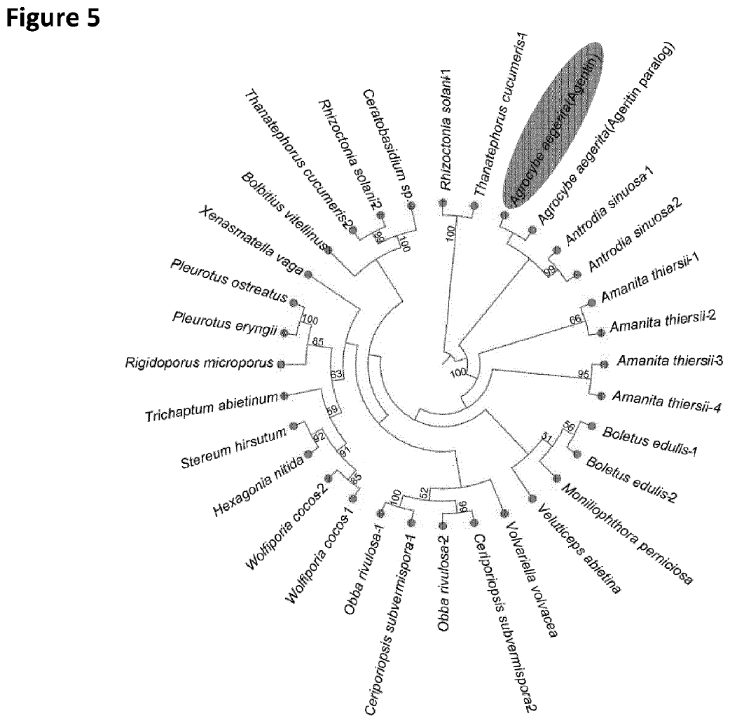

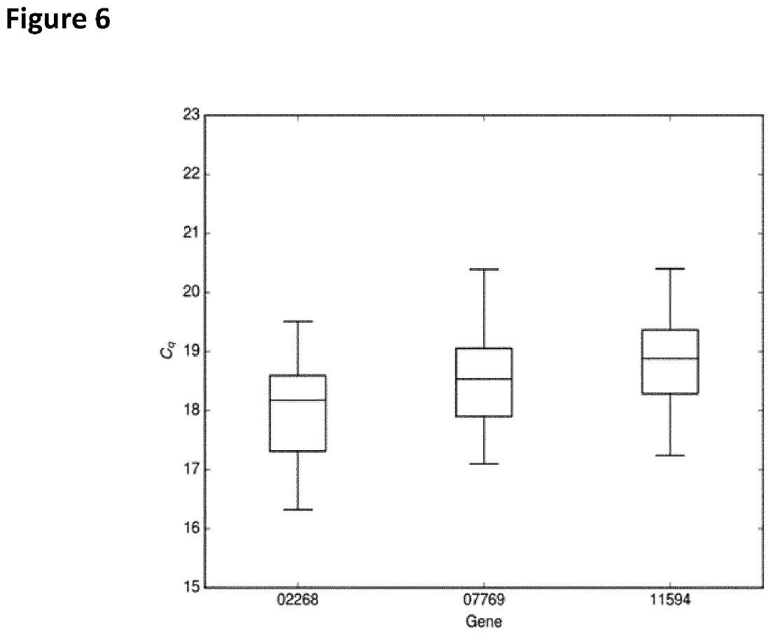

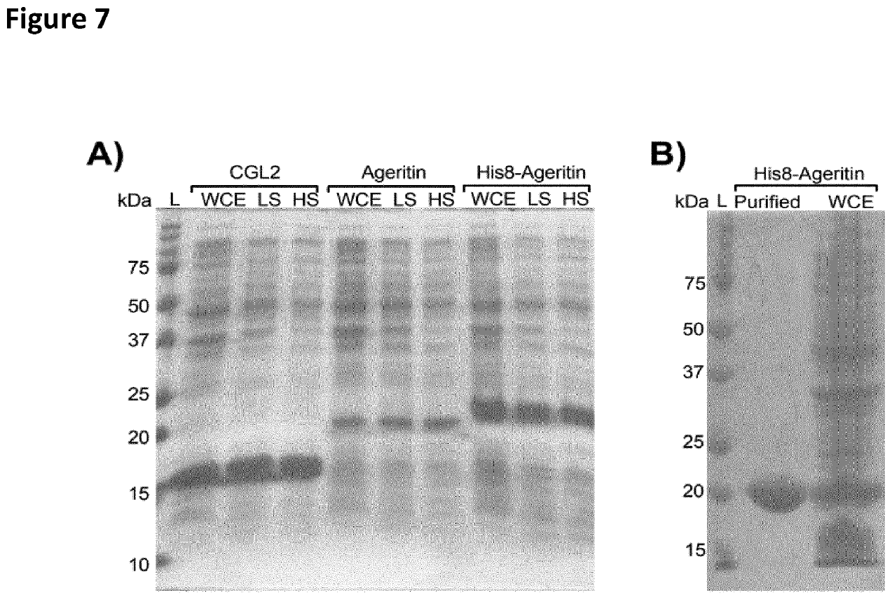

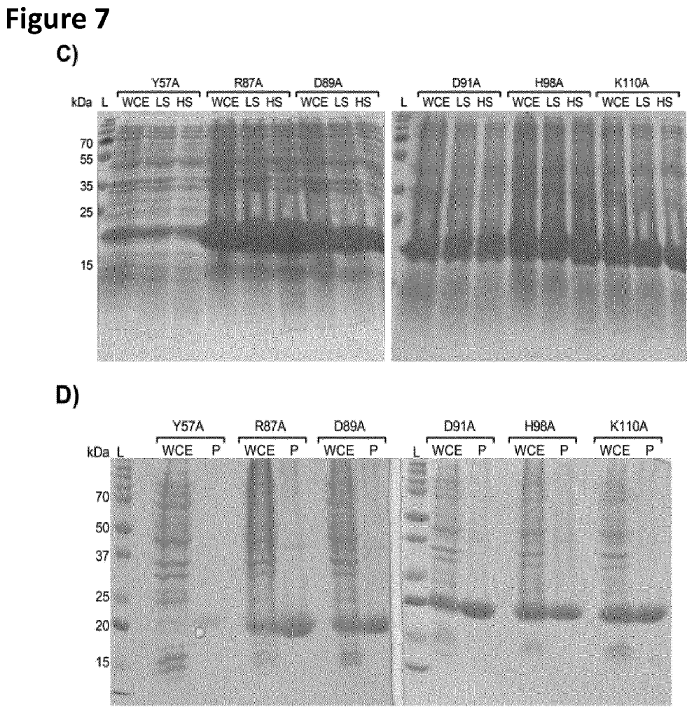

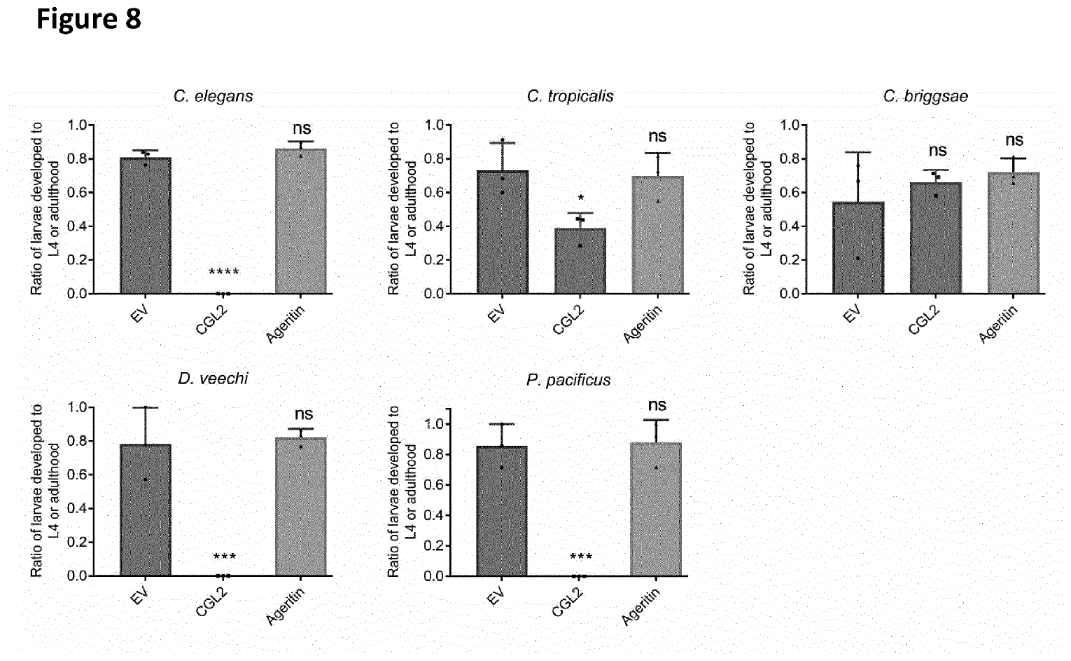

A) Amino acid sequences of ageritin and its paralog were aligned using the ClustalW algorithm (v2.1).

B) Expression level for the ageritin-encoding gene AaeAGT1 and the paralog AaeAGT2 at different stages of fruiting body development relative to that of vegetative mycelium (developmental stage I). The dotted horizontal line represents the mycelial expression level of both AaeAGT1 and AaeAGT2 and is used as a base-line value (developmental stage I), in comparison with other developmental stages of A. aegerita AAE-3: II, fruiting-primed mycelium 24 h to 48 h before emergence of visible fruiting body (FB) initials; III, FB initials; IV, entire FB primordia; Vs, young FB stipe; Vc, young FB cap. The error bars represent the standard deviation of three biological replicates.

C) Ribonucleolytic activity of ageritin assayed with 400 nM of purified ageritin against ribosomes of rabbit reticulocyte lysate. Ribotoxin α-Sarcin and reaction buffer were used as positive and negative control, respectively. Ribosomal RNAs and classical ribotoxin cleavage product, α-fragment, were indicated.

FIG. 2. Toxicity of ageritin against mosquito larvae and Spodoptera frugiperda Sf21 cells.

Entomotoxicity of ageritin and its tagged version was tested against Aedes aegypti larvae by recording the mortality (A) and by measuring the consumption of E. coli by determination of the reduction of the OD₆₀₀ of a respective E. coli suspension (B). Mosquito larvae were fed for 96 h on IPTG-induced E. coli BL21 expressing proteins Cgl2, ageritin and His₈-ageritin. E. coli BL21 cells expressing either previously characterized insecticidal protein Cgl2 or carrying ‘empty’ vector (EV) were used as positive and negative control, respectively. Statistical analysis was done using Dunnett's multiple comparisons test. The error bars represent the standard deviation of five biological replicates. ****p<0.0001 vs. EV.

C) Ageritin entomotoxicity was tested against the insect cell line S. frugiperda Sf21. Different concentrations of purified ageritin were incubated with Sf21 cells for 72 h. The number of viable cells was counted for each sample. DMSO and ribotoxin α-Sarcin were used as positive controls, and PBS buffer was used as a negative control. Dunnett's multiple comparisons test was used for statistical analysis. The error bars represent the standard deviation of six biological replicates. Symbols/abbreviations: ns: not significant, ***p<0.001, ****p<0.0001 vs. PBS.

FIG. 3. Effect of mutations in conserved residues on in vivo and in vitro activities of ageritin.

A) The amino acid sequence of ageritin was used as a query sequence for a BLAST search against the JGI MycoCosm fungal database. The hit regions of the top 10 sequences with highest homology were aligned using the ClustalW algorithm (v2.1). Individually mutated conserved regions are indicated by boxes and asterisks.

B)-D) The entomotoxic activity of of the wild-type (wt) and mutated ageritin versions was monitored by feeding Ae. aegypti larvae with E. coli BL21 cells expressing the respective protein of interest. Wild type and mutated ageritin toxicity was assessed by counting the number of surviving larvae (B), measuring OD₆₀₀-based bacteria consumption (C) every day for four days and counting number of larvae that reached the adult stage (D) by the end of day 7 of the larvae feeding on bacteria expressing one of the corresponding proteins. E. coli BL21 expressing either previously characterized insecticidal protein Cgl2 or carrying ‘empty’ vector (EV) were used as positive and negative controls, respectively. Statistical analysis was done using Dunnett's multiple comparisons test. The error bars represent the standard deviation of five biological replicates. ****p<0.0001 vs. EV

E) The ribonucleolytic activity of the ageritin wild-type and mutated ageritin proteins was assessed by exposing ribosomes of rabbit reticulocyte lysate to 400 nM the respective purified His₈-tagged protein. α-Sarcin and PBS buffer were used as positive and negative control, respectively. Ribosomal RNAs and ribotoxin cleavage product, α-fragment, were indicated.

FIG. 4. Functional comparison between ageritin and its paralog.

A) Heterologous expression and solubility of ageritin paralog in E. coli BL21. 20 μl of either bacterial whole cell extract (WCE), supernatants of WCE after low speed spin (LS; 5 min at 5000× g) or high speed spin (HS; 30 min at 16000× g) were loaded on a SDS-PAGE and stained with Coomassie brilliant blue.

B) Insecticidal activity of the ageritin paralog was tested against Ae. aegypti larvae by feeding the L3 staged mosquito larvae with E. coli BL21 expressing proteins of interests. Entomotoxic activity of the ageritin paralog against Ae. aegypti larvae. L3 mosquito larvae were fed E. coli BL21 bacteria expressing untagged and His₈-tagged versions of the ageritin paralog. Bacteria either expressing the previously characterized entomotoxic protein Cgl2 or carrying the ‘empty’ vector (EV) were used as positive and negative controls, respectively. The error bars represent the standard deviation from three biological replicates.

FIG. 5. Rooted circular cladogram of putative ageritin homologs.

The amino acid sequence of ageritin was used as a query sequence for a BLAST search against the database of the Gene Catalog Proteins (GCP) at the JGI MycoCosm fungal database. The complete amino acid sequence of top 30 hits were aligned using the ClustalW (v2.1), and phylogenetic relationships among the sequences were depicted via a circular cladogram. The hit with the lowest homology to ageritin among those 30 hits had an E-value of 7.2E⁻¹⁶, an identity of 44.8%, and a subject coverage of 64.8%. The branch lengths are relative and not to scale. Maximum likelihood bootstrap support values are indicated next to each node, if the bootstrap support values exceeded 50%. Different potential ageritin homologs in the genome of a given species are labeled by numbers.

FIG. 6. Expression levels of the reference genes for quantitative real-time reverse transcription-PCR.

AaeIMP1 (gene ID AAE3_02268), AaeTIF1 (gene ID AAE3_07769) and AaeARP1 (gene ID AAE3_11594) from the genome sequence of the dikaryon A. aegerita AAE-3 (Gupta et al., 2018), showing their transcription level by means of their Cq-values derived from quantitative real-time PCR analysis in a box-and-whisker-plot diagram. Whiskers indicate the variability outside the upper and lower quartile, respectively.

FIG. 7. Assessment of expression and solubility of ageritin in E. coli.

A) Heterologous expression and solubility of ageritin and its His₈-tagged version in E. coli BL21 cells. 20 μl of whole cell extract (WCE), supernatants of low speed spin (LS; 5 min. at 5000× g) and high speed spin (HS; 30 min. at 16000× g) of bacterial lysate were loaded on SDS-PAGE gel and stained with Coomassie brilliant blue. Cgl2 was used as a positive control for IPTG-induced expression and solubility analysis.

B) His₈-ageritin was produced in E. coli BL21 and 20 μl of Ni-NTA purified protein loaded onto the SDS-PAGE along with 20 μl of WCE and stained with Coomassie brilliant blue.

C) Ageritin single-site mutant constructs were produced in E. coli BL21, 20 μL, of WCE, LS and HS of each bacterial lysate were loaded onto an SDS-PAGE and stained with Coomassie brilliant blue. D) 5 μl of purified protein (P) of each mutant ageritin version were loaded on a SDS-PAGE along with 20 μl of its WCE and stained with Coomassie brilliant blue.

FIG. 8. Ageritin nematotoxicity tests.

Potential nematotoxicity of ageritin was tested against five different bacterivorous nematodes species: Caenorhadbitis elegans, C. briggsae, C. tropicalis, Distolabrellus veechi and Pristionchus pacificus. IPTG-induced E. coli BL21 expressing previously characterized nematotoxic protein Cgl2 or carrying ‘empty’ vector (EV) were used as positive and negative control, respectively. Dunnett's multiple comparisons test was used for statistical analysis. The error bars represent the standard deviation of three biological replicates. Symbols/abbreviations: ns: not significant, *p<0.05, **p<0.01, ***p<0.001, ****p<0.0001 vs. EV

### EXAMPLES

### Example

### Materials and Methods

### 1. Strains and Cultivation Conditions

Cultivation and strain maintenance of A. aegerita AAE-3 was performed as described previously (Herzog et al., 2016). Escherichia coli strains DH5α and E. coli BL21 (DE3) were used for cloning and protein expression, respectively. Other organisms including microorganisms used in this study are listed in Table 4.

TABLE 4
Organisms used in this study.

| Name | Strain | Source/Reference |
|---|---|---|
| Caenorhabditis | AF16 | Caenorhabditis Genetics Center (CGC) |
| briggsae | | |
| Caenorhabditis | N2 | Caenorhabditis Genetics Center (CGC) |
| elegans | | |
| Caenorhabditis | JU1373 | Caenorhabditis Genetics Center (CGC) |
| tropicalis | | |
| Distolabrellus | environ- | Luis Lugones, Utrecht University, |
| veechi | mental | Netherlands |
| | isolate | |
| Pristionchus | PS312 | Iain Wilson, BOKU, Vienna, Austria |
| pacificus | | |
| Aedes aegypti | Rockefeller | Pie Müller, Swiss Tropical and Public |
| | | Health Institute, Basel, Switzerland |
| Agrocybe aegerita | AAE-3 | Florian Hennicke, Senckenberg BiK-F, |
| | | Frankfurt a.M., Germany; genome- |
| | | sequenced (Gupta et al., 2018) |
| Escherichia coli | DH5α | |
| Escherichia coli | BL21(DE3) | Novagen |

### 2. Isolation of Total RNA from A. aegerita for Assessing Expression of AaeAGT1 and AaeAGT2

Fruiting induction for the sake of sampling different stages of fruiting body development of A. aegerita AAE-3 was performed as described by Herzog et al. (2016). In brief, 1.5% w/v malt extract agar (MEA) plates were inoculated centrally with a 0.5 cm diameter agar plug originating from the growing edge of an A. aegerita AAE-3 culture. Before fruiting induction (20° C., 12 h/12 h light/dark cycle, saturated humidity, aeration, local injury of the mycelium by punching out a 0.5 cm diameter mycelium-overgrown MEA plug), fungal plates were incubated for ten days at 25° C. in the dark.

Samples, consisting of at least three independent replicates, were retrieved from the following developmental stages: I) vegetative mycelium prior to fruiting induction at day ten post inoculation; II) fruiting-primed mycelium 24 h to 48 h before emergence of fruiting body initials at day 14 post inoculation; III) fruiting body initials at day 15 to 16 post inoculation; IV) fruiting body primordia at day 17 to the morning of day 19 post inoculation; Vs-Vc) young fruiting bodies separated into stipe (Vs) and cap (Vc) plectenchyme at day 19 to the morning of day 21 post inoculation. Mature fruiting bodies exhibiting full cap expansion and a spore print emerging by morning of day 22 post inoculation were not sampled. Sample I) and II) were obtained by gently scraping off the outermost 1 cm of mycelium from three replicate agar plates by gently scraping with a sterile spatula. Samples were transferred immediately to a 2 mL microcentrifuge tube containing 1 mL of RNAlater® (product ID: R0901, Sigma Aldrich GmbH, Munich, Germany) which was transferred to 4° C. for a maximum of 3 days before freezing at −80° C. until total RNA extraction.

For total RNA extraction, NucleoSpin® RNA Plant kit (product ID: 740949, Macherey-Nagel GmbH & Co. KG, Düren, Germany) was used whereby cell homogenization and lysis were modified. First, the RNAlater® was removed from each pooled sample and an appropriate amount of lysis Buffer RA1 added (350 μl per 100 mg fungal biomass). One 4 mm- and about ten 1 mm-diameter acetone-cleaned stainless-steel beads (product IDs: G40 and G10, respectively, KRS-Trading GmbH, Barchfeld-Immelborn, Germany) were then added to each tube. Homogenization was achieved using a mixer mill MM 200 (Retsch, Haan, Germany) set to 8 min at 25 Hz. Then, the protocol followed the recommendations of the manufacturer for RNA-extraction from filamentous fungi, including a DNA digestion step with the kit's rDNase, beginning with the “filtrate lysate” step.

Total RNA was eluted in nuclease-free water (product ID: T143, Carl Roth GmbH & Co. KG, Karlsruhe, Germany) and the RNA concentration was measured spectrophotometrically with a NanoDrop 2000 c (Thermo Fisher Scientific, Waltham, USA). RNA quality was visually assessed by checking the integrity of the major rRNA bands in a denaturing polyacrylamide gel (Urea-PAGE). Per lane, 1 μg of total RNA was loaded onto pre-cast Tris-borate-EDTA (TBE)-urea 6% polyacrylamide gels (product ID: EC68652BOX, Thermo Fischer Scientific, Waltham, USA) and separated for 1 h at a constant voltage of 180V. For detection, SYBR™ Gold (product ID: S11494, Thermo Fisher Scientific) was used to stain the gel following the manufacturer's recommendations. Only if no degradation of the RNA was observed, with major rRNA bands intact, the respective sample was further processed. Total RNAs were routinely stored at −80° C.

### 3. Determination of Suitable Reference Genes for Quantitative Real-Time Reverse Transcription-PCR

Primer pairs for A. aegerita AAE-3 genes AaeIMP1 (gene ID AAE3_02268), AaeTIF1 (gene ID AAE3_07769) and AaeARP1 (gene ID AAE3_11594) have been designed manually on the basis of their genomic DNA (Gupta et al., 2018). In each case, the gene name was assigned in accordance with the name of the putative encoded protein according to the UniProt database (www.uniprot.org). Each primer pair (Table 5, see below) spans a cDNA nucleotide region of 150 bp. Twelve reference samples were taken from vegetative mycelia grown for 10, 14, 18, 20, 22, 24 and 27 days on agar plates, from a primordia source of 18 day old cultures, and from fruiting bodies before (two samplings on different days from young fruiting bodies), during (one sampling) and after sporulation (one sampling). The samples were stored in RNAlater® (product ID: 76104, Qiagen, Venlo, Netherlands). Total RNA was extracted using TRIzol (Thermo Fischer Scientific) by the method of Chomczynski and Sacchi (1987). The RNA concentration was determined by the absorbance at 260 nm using a Pearl Nanophotometer (Implen, Munich, Germany). 2 μg total RNA of each sample was reverse transcribed applying the M-MLV reverse transcriptase kit according to the manufacturer's protocol (Thermo Fischer Scientific) using oligo-(dT)₃₀-Primer. The resulting cDNA sample was incubated for 20 min at 37° C. with 1 μL AMRESCO RNase A (VWR International, Radnor, Pa., USA) instead of the RNase H described in the M-MLV reverse transcriptase kit protocol. Quantitative real-time polymerase chain reaction was conducted in triplicates using the KAPA SYBR® FAST Universal Kit (Sigma Aldrich GmbH) according to the manufacturer's protocol with an annealing step at 60° C. for 30 sec, an elongation step at 72° C. for 10 sec in a total volume of 25 μL with a final concentration of 0.9 μM for each primer and 7 μL of cDNA template on a CFX connect Real-Time Detection System (Bio-Rad, Hercules, Calif., USA).

To test primer efficiency, equal aliquots from each cDNA template were combined. Six logarithmic dilution steps of the cDNA master mix were used for qPCR (see above). Cq-values of the dilutions were blotted versus the dilution factor and linear regression as well as the corresponding determination coefficient was calculated for each reference gene. The primer efficiency was calculated according to Pfaffl (2001). For validation of the reference genes, all 12 cDNA samples were used separately for a quantitative real-time PCR analysis with all three primer pairs. Cq-values were used for validation using the NormFinder (Andersen et al., 2004) and geNorm (Vandesompele et al., 2002) algorithm.

### 4. One-Step Quantitative Real-Time Reverse Transcription-PCR (qRT-PCR) Using Total RNA Samples

Primers and qRT-PCR conditions were designed according to the general recommendations of the MIQE guidelines (Bustin et al., 2009). The software Geneious R11 (https://www.geneious.com, Kearse et al. (2012) was applied to design the primers for the genes encoding ageritin (AaeAGT1, gene ID AAE3_01767) and its paralog (AaeAGT2, gene ID AAE3_01768) as well as the two reference genes AaeTIF1 (gene ID AAE3_07669) and AaeIMP1 (gene ID AAE3_02268) from the A. aegerita AAE-3 genome sequence (Gupta et al., 2018; see also www.thines-lab.senckenberg.de/agrocybe_genome). Primers for these genes are also listed in Table 5.

All qRT-PCRs were performed on an AriaMX Real-Time PCR System (Agilent Technologies, La Jolla, Calif., USA) in optically clear 96-well plates with 8-cap strips using the Brilliant Ultra-Fast SYBR Green QRT-PCR Master Mix (Agilent Technologies). This one-step master mix included the reverse transcriptase (RT), the reaction buffer, the DNA polymerase and SYBR green. The RT-reaction was performed in each well prior to the start of the qRT-PCR program. All samples were run in three biological replicates with 25 ng total RNA per reaction mixture. The final concentration per primer was 250 nM. A melting curve analysis was done for each reaction at the end of the qRT-PCR to determine amplicon purity. See Table 6 for the qRT-PCR program.

TABLE 5
Primers used in this study.

| | | SEQ |
|---|---|---|
| Primer^{a} | Sequence 5′-3′^{b} | ID NO. |
| pAGT1-Nd | ggcgcatATGTCCGAGTCCTCTACCTTCACCACTGC | 5 |
| pAGT1-N | gtgcggccgcTCACGCCGGAGCCTTGCCC | 6 |
| pF_8His-Ag | CACCACCACCACGAGTCCTCTACCTTCACCACTG | 7 |
| pR_8His-Ag | ATGATGATGATGGGACATATGTATATCTCCTTCT | 8 |
| HAgP-FW | ggggggcatATGAGCCATCATCATCATCACCACCACC | 33 |
| | ACGACCCGAGCGCGCCGGG | |
| AgP-RV | ggggggctcgagtgcggccgcTTACGCCGGCGCTTTG | 34 |
| pF_8His-Ag(Y57A) | AAGTTGGTCACGGCCACCAGCCGCC | 9 |
| pR_8His-Ag(Y57A) | GGTcrrATcAATTTTGTccrrccccG | 10 |
| pF_8His-Ag(R87A) | GTCGCGCTCGACATGGACAACACC | 11 |
| pR_8His-Ag(R87A) | GTAGGGCACGATGGAGCCCGCGGC | 12 |
| pF_8His-Ag(D89A) | TGCCCTACGTCCGGCTCGCCATGG | 13 |
| pR_8His-Ag(D89A) | CGATGGAGCCCGCGGCCGTTTTGA | 14 |
| pF_8His-Ag(D91A) | GTCCGGCTCGACATGGCCAACACC | 15 |
| pR_8His-Ag(D91A) | GTAGGGCACGATGGAGCCCGCGGC | 16 |
| pF_8His-Ag(H98A) | GGCAAGGGCATCGCTTTCAA | 17 |
| pR_8His-Ag(H98A) | GGTGTTGTCCATGTCGAGCC | 18 |
| pF_8His-Ag(K110A) | AGTTCCGCCGCGCTCGCCG | 19 |
| pR_8His-Ag(K110A) | GTCGGAGAGTTTAGTCGCGTTGAAA | 20 |
| cds017674 | TTCTTTTCGCTACTCAGAATCGTTG | 21 |
| cds01767-r | CAGAGCTCTCCCAACCACAG | 22 |
| 02268_f | AGATGCGTATTCTGATGGTTGGTC | 23 |
| 02268_r | CCCACACTGTGAATGAGATGTTC | 24 |
| 07769_f | ATTCCTACGATCCTTTTGCCG | 25 |
| 07769_r | GATcATATTGrrTcGGGAGTccT | 26 |
| 11594_f | TCTGATCTGACTGTCGGCCAA | 27 |
| 11594_r | ATCCTCGTCCTTATGCTCCTC | 28 |
| 01767_f | AAGCCCCGCATATCAGAAG | 29 |
| 01767_r | CTGTCGGAGAGTTTAGTCGC | 30 |
| 01768_f | GAAAGACCCAGATTGACCCAG | 31 |
| 01768_f | GTGAATTTTAGGCCGACGC | 32 |

^{a}Primers used for quantitative real-time PCR procedures are labelled with forward (f) and reverse (r), respectively.
Primers for cDNA generation start by coding sequence (cds) and end by forward (fw) or reverse (rv).
^{b}Lowercase letters are primer extensions to create the underlined restriction sites.

TABLE 6
qRT-PCR program for measuring AaeAGT1 and AaeAGT2 expression
in this study.

| Programme step | | Temperature | Time |
|---|---|---|---|
| Reverse transcription | | 50° C. | 10 min |
| Initial denaturation (hot start) | | 95° C. | 3 min |
| 40 cycles | Denaturation | 95° C. | 5 sec |
| | Annealing | 58° C. | 10 sec |
| | Extension | 72° C. | 10 sec |

| | | |
|---|---|---|
| Melting curve | 65° C. to 95° C. | 5 min |
| | (resolution 0.5° C.) | |

### 5. Relative Differential Expression Analysis

Data analysis was based on Cq values calculated from raw fluorescence intensities. The baseline correction and determination of the quantification cycle (Cq) and mean PCR efficiency (E) per amplicon was done according to Ruijter et al. (2009) using the LinRegPCR program in version 2017.1.

Relative expression ratios were calculated using the software REST, version REST2009 (Pfaffl et al. 2002). It employs the ‘Pfaffl method’ (Pfaffl, 2001) to calculate the E corrected relative gene expressions ratios, allowing for the simultaneous use of multiple reference genes for normalization based on Vandesompele et al. (2002). 95% confidence intervals around the mean relative expression ratios were calculated on the basis of 2,000 iterations. Vegetative mycelium that was not induced for fruiting (developmental stage I) was chosen as calibration sample.

### 6. cDNA Generation from A. aegerita RNA

The cDNA was synthetized from total RNA of fruiting-primed mycelium using the RevertAid first strand cDNA synthesis kit (product ID: K1621, Thermo Fisher Scientific) and an oligo(dT)₁₈ primers. First strand total-cDNA was then directly used as a template to produce the specific cDNA of the ageritin-encoding gene AaeAGT1 (gene ID AAE3_01767) with primer pair cds01767-f and cds01767-r (see Table 5) in a standard 3-step PCR using Phusion polymerase (product ID: F530, Thermo Fischer Scientific) with a annealing temperature of 62° C. DNA sequence information for primer design was obtained from the genome sequence of A. aegerita AAE-3 (Gupta et al., 2018).

verified cDNA sequence of gene AaeAGT1, gene ID
AAE3_01767:
SEQ ID NO. 2
atgtccgagtcctctaccttcaccactgcggtagtacctgaaggcgaa
ggagttgctccaatggcagagaccgtgcagtattacaactcctactct
gacgcatccatcgcgtcttgcgcatttgtagactcggggaaggacaaa
attgataagaccaagttggtcacgtacaccagccgcctcgccgcaagc
cccgcatatcagaaggtcgtcggcgtcggcctcaaaacggccgcgggc
tccatcgtgccctacgtccggctcgacatggacaacaccggcaagggc
atccatttcaacgcgactaaactctccgacagttccgccaagctcgcc
gcggtgctcaagacgacggtgtccatgaccgaggcacagcgaactcaa
ctctacatggagtatatcaagggcatcgagaatcggagtgcgcagttt
atttgggactggtggaggacgggcaaggctccggcgtga

### 7. Construction of Ageritin Expression Vectors

The coding sequence of ageritin was identified by BLAST analysis using the published 25 N-terminal residues of ageritin against the predicted proteome of Agrocybe aegerita (Gupta et al., 2018). The sequence was amplified with the primer pair pAGT1-Nd and pAGT1-N (see Table 5) from the AaeAGT1-cDNA and cloned into a pET-24b (+) expression vector. The sequence of the cloned cDNA was confirmed by DNA sequencing. The plasmid was transformed into E. coli BL21 cells. For expression of ageritin, E. coli BL21-transformants were pre-cultivated in Luria-Bertani (LB) medium supplemented with 50 mg/1 kanamycin at 37° C. At an OD₆₀₀ of around 0.5, the cells were induced with 0.5 mM isopropyl ↑-D-1-thiogalactopyranoside (IPTG) (product ID: I8000, BioSynth AG, Switzerland) and cultivated over night at 16° C. Expression and solubility of ageritin was checked as previously described (Künzler et al., 2010).

### 8. Toxicity Against Mosquito Larvae and Nematodes

Egg masses of the yellow fever mosquito, Aedes aegypti, were harvested on filter papers from the Rockefeller laboratory colony reared at the Swiss Tropical and Public Health Institute (Basel, Switzerland). For the experiments, mosquito larvae were reared by placing 2- to 5-cm² small pieces of the egg paper, depending on the density of the eggs, into glass petri dishes containing tap water at 28° C. The larvae hatched within a few hours. They were fed with finely ground commercially available food for ornamental fish.

The toxicity assays against the mosquito larvae were performed as described previously (Künzler et al., 2010). In brief, larvae in their third stage (L3) were used for the toxicity assays. The food source was changed from fish food to E. coli, adjusted in all the bioassays to an OD₆₀₀ of 0.4. The mosquito larvae fed readily on E. coli and were able to develop to the adult stage. The consumption of E. coli bacteria carrying the empty vector by the mosquito larvae could be tracked by the reduction in the optical density. The bioassay was performed by transferring ten L3 larvae to Schott bottles containing 99 mL of tap water and 1 ml of E. coli cells expressing the desired protein. The mosquito larvae were kept in the dark at 28° C. and the toxicity was assessed by the larval mortality over the first four days and by the reduction in optical density. In addition, the number of adult mosquitoes, which were able to develop from the larvae, was counted after 7 days. The previously characterized entomo- and nematotoxic lectin Cgl2 was used as a positive control in all toxicity assays (Bleuler-Martinez et al., 2011). Starvation controls were used to check whether the death of the larvae is due to toxicity or refrainment of the larvae from consuming the bacteria. Nematotoxicity assays against five different species of nematodes were performed as described before (Künzler et al., 2010; Plaza et al., 2016). Dunnett's multiple comparisons test was used to calculate the statistical differences between mean values of treatment and control groups.

### 9. Tagging and Purification of Ageritin

For the purification of recombinant ageritin over Ni-NTA columns (Macherey-Nagel), the protein was tagged with a polyhistidine(His₈)-tag at its N-terminus. A plasmid was constructed by PCR using pF_8His-Ag and pR_8His-Ag primer pair listed in Table 5. His₈-ageritin was expressed as described for untagged ageritin. Protein purification was performed as described previously (Bleuler-Martinez et al., 2011) but by using Tris-HCl, pH 7.5, as the lysis, purification, and storage buffer.

### 10. In Vitro rRNA Cleavage Assay

To detect the rRNA cleavage activity, 20 μL of untreated rabbit reticulate lysate (product ID: L4151, Promega, Wis., USA) was mixed with a final concentration of 400 nM ageritin or with the mutant versions in the reaction buffer (15 mM Tris-HCl, 15 mM NaCl, 50 mM KCl, 2.5 mM EDTA. pH 7.6) to a final volume of 30 μL (Kao et al., 2001). The reaction mixture was incubated for one hour at 30° C., and stopped by adding 3 μL of 10% SDS. RNA was isolated from the reaction mixture by chloroform-phenol extraction. The RNAs were mixed with 2× RNA loading dye (Thermo Fisher Scientific) and denatured for 5 minutes at 65° C., cooled on wet ice and run on 2% native agarose gel in cold TBE Buffer (Tris-borate-EDTA) for 30 minutes at 100 V. α-Sarcin (product ID: BCO-5005-1, Axxora, USA) and phosphate-buffered saline (PBS) buffer were used as positive and negative control, respectively.

### 11. Toxicity Towards Insect Cells

Cytotoxicity of ageritin was tested against the insect cell line of Spodoptera frugiperda Sf21 (IPLB-Sf21-AE). The insect cells were pre-cultivated in Sf-900TM II serum-free medium (SFM; Invitrogen, Calif., USA) supplemented with streptomycin (100 μg/mL) and penicillin (100 μg/mL). Cells were diluted to a final density of 0.29×10⁶/mL and 500 μL of per well were dispensed in 24-well plates. The insect cells were challenged with different concentrations of ageritin (0.1 μM, 1 μM and 10 μM) dissolved in PBS. The well plates were incubated for three days at 27° C. The liquid medium was removed, and the cells were stained with 15 μl of 0.4% trypan blue solution. The number of alive and thus unstained cells was determined under a microscope. α-Sarcin (Axxora) and 5% DMSO were used as positive controls whereas the PBS buffer served as a negative control. Dunnett's multiple comparisons test was used to test whether the observed differences between the mean values of the treatment and control groups are statistically significant.

### 12. Alignment and Phylogenetic Tree

The complete amino acid sequence of ageritin was used as a query in a BLAST search against the database of the Gene Catalog Proteins (GCP) at JGI MycoCosm (Grigoriev et al., 2014). The hit regions of the top ten sequences with highest homology were aligned using the ClustalW algorithm (v2.1) at CLC Genomics Workbench (Chenna et al., 2003).

For the analysis of the phylogenetic relationship among the top homologs of ageritin, the complete amino acid sequences of 30 hits were aligned using ClustalW (v2.1). A phylogenetic tree was constructed employing the Maximum Likelihood algorithm (Aldrich, 1997). The tree was designed as a rooted circular cladogram. The hit with the lowest homology to ageritin among the 30 hits had an E-value of 7.2E-16.

### 13. Creation and Expression of Mutant Versions

Based on the alignment, six of the completely conserved residues (Y57, R87, D89, D91, H98 and K110) of ageritin were mutated individually to alanine using the site-specific primers listed in Table 5. The plasmid encoding His₈-ageritin was used as template for construction of single-site mutants. Expression and purification of the mutant ageritin variants were done as for wild type His₈-ageritin.

### 14. Expression and Purification of Ageritin Paralog

The predicted coding sequence for the paralog of ageritin (63% sequence identity) was ordered in a codon-optimized version (for E. coli) from GenScript (Piscataway, N.J., USA). Expression and purification of the paralogous protein was done as described above for for wild-type His₈-ageritin.

The features disclosed in the foregoing description, in the claims and/or in the accompanying drawings may, both separately and in any combination thereof, be material for realizing the invention in diverse forms thereof.

### REFERENCES

Aldrich J: R. A. Fisher and the making of maximum likelihood 1912-1922. Stat Sci 1997, 12(3):162-176.

Andersen C L, Jensen J L, Orntoft T F: Normalization of real-time quantitative reverse transcription-PCR data: A model-based variance estimation approach to identify genes suited for normalization, applied to bladder and colon cancer data sets. Cancer Res 2004, 64(15):5245-5250.

Barzon L. Ongoing and emerging arbovirus threats in Europe. J Clin Virol. 2018 October; 107:38-47. doi: 10.1016/j.jcv.2018.08.007.

Berry et al. Complete sequence and organization of pBtoxis, the toxin-coding plasmid of Bacillus thuringiensis subsp. israelensis. Appl Environ Microbiol. 2002; 68:5082-95.

Bleuler-Martinez S, Butschi A, Garbani M, Walti M A, Wohlschlager T, Potthoff E, Sabotic J, Pohleven J, Luthy P, Hengartner M O et al: A lectin-mediated resistance of higher fungi against predators and parasites. Mol Ecol 2011, 20(14):3056-3070.

Bolognesi A, Bortolotti M, Maiello S, Battelli M G, Polito L: Ribosome-Inactivating Proteins from Plants: A Historical Overview. Molecules 2016, 21(12).

Bustin S A, Benes V, Garson J A, Hellemans J, Huggett J, Kubista M, Mueller R, Nolan T, Pfaffl M W, Shipley G L et al: The MIQE Guidelines: Minimum Information for Publication of Quantitative Real-Time PCR Experiments. Clin Chem 2009, 55(4):611-622.

Cantón P E, Zanicthe Reyes E Z, Ruiz de Escudero I, Bravo A, Soberón M. Binding of Bacillus thuringiensis subsp. israelensis Cry4Ba to Cyt1Aa has an important role in synergism. Peptides. 2011 March; 32(3):595-600. doi: 10.1016/j.peptides.2010.06.005. Epub 2010 Jun. 15.

Cadavid-Restrepo G, Sahaza J, Orduz S. Treatment of an Aedes aegypti colony with the Cry11Aa toxin for 54 generations results in the development of resistance. Mem Inst Oswaldo Cruz. 2012 February; 107(1):74-9.

Chan Y L, Endo Y, Wool I G: The Sequence of the Nucleotides at the Alpha-Sarcin Cleavage Site in Rat 28-S Ribosomal Ribonucleic-Acid. J Biol Chem 1983, 258(21):2768-2770.

Cheng et al., 2004, Chemical composition and mosquito larvicidal activity of essential oils from leaves of different Cinnamomum osmophloeum provenances. J Agric Food Chem 52:4395-4400.

Cheng et al., 2009, Insecticidal activities of leaf essential oils from Cinnamomum osmophloeum against three mosquito species. Bioresour Technol. 100:457-464.

Chenna R, Sugawara H, Koike T, Lopez R, Gibson T J, Higgins D G, Thompson J D: Multiple sequence alignment with the Clustal series of programs. Nucleic Acids Research 2003, 31(13):3497-3500.

Chomczynski P, Sacchi N: Single-Step Method of Rna Isolation by Acid Guanidinium Thiocyanate Phenol Chloroform Extraction. Anal Biochem 1987, 162(1):156-159.

Cloonan K R, Andreadis S S, Chen H, Jenkins N E, Baker T C. Attraction, Oviposition and Larval Survival of the Fungus Gnat, Lycoriella ingenua, on Fungal Species Isolated from Adults, Larvae, and Mushroom Compost. PLoS One. 2016 Dec. 9; 11(12):e0167074. doi: 10.1371/journal.pone.0167074. eCollection 2016.

Correll C C, Munishkin A, Chan Y L, Ren Z, Wool I G, Steitz T A: Crystal structure of the ribosomal RNA domain essential for binding elongation factors. P Natl Acad Sci USA 1998, 95(23): 13436-13441.

Endo Y, Tsurugi K, Yutsudo T, Takeda Y, Ogasawara T, Igarashi K: Site of action of a Vero toxin (VT2) from Escherichia coli 0157:H7 and of Shiga toxin on eukaryotic ribosomes. RNA N-glycosidase activity of the toxins. Eur J Biochem 1988, 171(1-2):45-50.

Gupta D K, Rühl M, Mishra B, Kleofas V, Hofrichter M, Herzog R, Pecyna M J, Sharma R, Kellner H, Hennicke F et al: The genome sequence of the commercially cultivated mushroom Agrocybe aegerita reveals a conserved repertoire of fruiting-related genes and a versatile suite of biopolymer-degrading enzymes. Bmc Genomics 2018, 19.

Grigoriev I V, Nikitin R, Haridas S, Kuo A, Ohm R, Otillar R, Riley R, Salamov A, Zhao X L, Korzeniewski F et al: MycoCosm portal: gearing up for 1000 fungal genomes. Nucleic Acids Research 2014, 42(D1):D699-D704.

Herrero-Galan E, Lacadena J, del Pozo A M, Boucias D G, Olmo N, Onaderra M, Gavilanes J G: The insecticidal protein hirsutellin A from the mite fungal pathogen Hirsutella thompsonii is a ribotoxin. Proteins 2008, 72(1):217-228.

Herrero-Galán E, Álvarez-García E, Carreras-Sangrà N, Lacadena J, Alegre-Cebollada J, Martínez del Pozo Á, Oñaderra M, Gavilanes J G: Fungal ribotoxins: structure, function and evolution. In: Microbial Toxins: Current Research and Future Trends. 2009: 167-187.

Herrero-Galán E, García-Ortega L, Lacadena J, Martínez-Del-Pozo Á, Olmo N, Gavilanes J G, Oñaderra M: Implication of an Asp residue in the ribonucleolytic activity of hirsutellin A reveals new electrostatic interactions at the active site of ribotoxins. Biochimie 2012, 94:427-433.

Herrero-Galán E, García-Ortega L, Olombrada M, Lacadena J, Del Pozo Á M, Gavilanes J G, Oñaderra M: Hirsutellin A: A Paradigmatic Example of the Insecticidal Function of Fungal Ribotoxins. Insects 2013, 4:339-356.

Herzog R, Solovyeva I, Ruhl M, Thines M, Hennicke F: Dikaryotic fruiting body development in a single dikaryon of Agrocybe aegerita and the spectrum of monokaryotic fruiting types in its monokaryotic progeny. Mycol Prog 2016, 15(9):947-957.

Kampen H, Schuhbauer A, Walther D: Emerging mosquito species in Germany-a synopsis after 6 years of mosquito monitoring (2011-2016). Parasitol Res 2017, 116(12):3253-3263.

Kao R, Martínez-Ruiz A, Del Pozo A M, Crameri R, Davies J: Mitogillin and related fungal ribotoxins. Methods in Enzymology 2001, 341:324-335.

Kearse M, Moir R, Wilson A, Stones-Havas S, Cheung M, Sturrock S, Buxton S, Cooper A, Markowitz S, Duran C et al: Geneious Basic: An integrated and extendable desktop software platform for the organization and analysis of sequence data. Bioinformatics 2012, 28(12): 1647-1649.

Künzler M, Bleuler-Martinez S, Butschi A, Garbani M, Lüthy P, Hengartner M O, Aebi M: Biotoxicity assays for fruiting body lectins and other cytoplasmic proteins. 2010, 480:141-150.

Lacadena J, del Pozo A M, Martinez-Ruiz A, Perez-Canadillas J M, Bruix M, Mancheno J M, Onaderra M, Gavilanes C G: Role of histidine-50, glutamic acid-96, and histidine-137 in the ribonucleolytic mechanism of the ribotoxin alpha-sarcin. Proteins-Structure Function and Genetics 1999, 37(3):474-484.

Lacadena J, Alvarez-Garcia E, Carreras-Sangra N, Herrero-Galan E, Alegre-Cebollada J, Garcia-Ortega L, Onaderra M, Gavilanes J G, del Pozo A M: Fungal ribotoxins: molecular dissection of a family of natural killers. Fems Microbiol Rev 2007, 31(2):212-237.

Landi N, Pacifico S, Ragucci S, Iglesias R, Piccolella S, Amici A, Di Giuseppe A M A, Di Maro A: Purification, characterization and cytotoxicity assessment of Ageritin: The first ribotoxin from the basidiomycete mushroom Agrocybe aegerita. Biochimica et Biophysica Acta-General Subjects 2017, 1861:1113-1121.

Olombrada M, Herrero-Galán E, Tello D, Oñaderra M, Gavilanes J G, Martínez-Del-Pozo Á, García-Ortega L: Fungal extracellular ribotoxins as insecticidal agents. Insect Biochem Mol Biol 2013, 43 39-46.

Olombrada M, Martínez-Del-Pozo Á, Medina P, Budia F, Gavilanes J G, García-Ortega L: Fungal ribotoxins: Natural protein-based weapons against insects. Toxicon 2014, 83:69-74.

Olombrada M, Medina P, Budia F, Gavilanes J G, Martinez-Del-Pozo A, Garcia-Ortega L: Characterization of a new toxin from the entomopathogenic fungus Metarhizium anisopliae: the ribotoxin anisoplin. Biol Chem 2017, 398(1):135-142.

Olombrada M, Lázaro-Gorines R, López-Rodríguez J, Martínez-del-Pozo Á, Oñaderra M, Maestro-López M, Lacadena J, Gavilanes J, García-Ortega L: Fungal Ribotoxins: A Review of Potential Biotechnological Applications. Toxins 2017, 9:71.

Plaza D F, Schmieder S S, Lipzen A, Lindquist E, Künzler M: Identification of a Novel Nematotoxic Protein by Challenging the Model Mushroom Coprinopsis cinerea with a Fungivorous Nematode. Genes|Genomes|Genetics 2016, 6:87-98.

Perez-Canadillas J M, Santoro J, Campos-Olivas R, Lacadena J, del Pozo A M, Gavilanes J G, Rico M, Bruix M: The highly refined solution structure of the cytotoxic ribonuclease alpha-sarcin reveals the structural requirements for substrate recognition and ribonucleolytic activity. Journal of Molecular Biology 2000, 299(4):1061-1073.

Pfaffl M W: A new mathematical model for relative quantification in real-time RT-PCR. Nucleic Acids Research 2001, 29(9).

Pfaffl M W, Horgan G W, Dempfle L: Relative expression software tool (REST (c)) for group-wise comparison and statistical analysis of relative expression results in real-time PCR. Nucleic Acids Research 2002, 30(9).

Ruijter J M, Ramakers C, Hoogaars W M H, Karlen Y, Bakker O, van den Hoff M J B, Moorman A F M: Amplification efficiency: linking baseline and bias in the analysis of quantitative PCR data. Nucleic Acids Research 2009, 37(6).

Sawa T, Hamaoka S, Kinoshita M, Kainuma A, Naito Y, Akiyama K, Kato H: Pseudomonas aeruginosa Type III Secretory Toxin ExoU and Its Predicted Homologs. Toxins 2016, 8(11).

Suter T, Crespo M M, de Oliveira M F, Alves de Oliveira M A, Varjal de Melo-Santos C M, Fontes de Oliveira C M, Junqueira Ayres C F, Rodrigues Barbosa R M, Araújo A P, Regis L N, Flacio E, Engeler L, Müller P, Neves Lobo Silva-Filha M H: Insecticide susceptibility of Aedes albopictus and Ae. aegypti from Brazil and the Swiss-Italian border region. Parasites & Vectors 2017, 10:431.

Vandesompele J, De Preter K, Pattyn F, Poppe B, Van Roy N, De Paepe A, Speleman F: Accurate normalization of real-time quantitative RT-PCR data by geometric averaging of multiple internal control genes. Genome Biology 2002, 3(7).

Weeks E N, Baniszewski J, Gezan S A, Allan S A, Cuda J P, Stevens B R. Methionine as a safe and effective novel biorational mosquito larvicide. Pest Manag Sci. 2018 Jun. 11. doi: 10.1002/ps.5118.

Yang X J, Moffat K: Insights into specificity of cleavage and mechanism of cell entry from the crystal structure of the highly specific Aspergillus ribotoxin, restrictocin. Structure 1996, 4(7):837-852.

Yao Q Z, Yu M M, Ooi L S M, Ng T B, Chang S T, Sun S S M, Ooi V E C: Isolation and characterization of a type 1 ribosome-inactivating protein from fruiting bodies of the edible mushroom (Volvariella volvacea). J Agr Food Chem 1998, 46(2):788-792.

1-17. (canceled)

18. A protein selected from
a protein comprising an amino acid sequence of SEQ ID NO: 1 or an amino acid sequence having at least 60% sequence identity to the amino acid sequence of SEQ ID NO: 1, and/or
a protein encoded by a nucleotide sequence of SEQ ID NO: 2 or a nucleotide sequence having at least 60% sequence identity to the nucleotide sequence of SEQ ID NO: 2.

19. The protein of claim 18, comprising an amino acid substitution in position Y57, D91 and/or K110.

20. A nucleic acid molecule, comprising a nucleotide sequence of SEQ ID NO: 2 or a nucleotide sequence having at least 60% sequence identity to the nucleotide sequence of SEQ ID NO: 2.

21. The nucleic acid molecule of claim 20, further comprising vector nucleic acid sequences, and/or comprising promoter nucleic acid sequences and terminator nucleic acid sequences, and/or comprising other regulatory nucleic acid sequences, and/or wherein the nucleic acid molecule comprises dsDNA, ssDNA, cDNA, LNA, PNA, CNA, RNA or mRNA or combinations thereof.

22. A host cell, containing a nucleic acid molecule according to claim 20.

23. The host cell of claim 22, which is a bacterial cell
a plant cell,
or
a fungal cell.

24. A recombinant protein of claim 18 obtained from a host cell containing a nucleic acid molecule encoding the protein of claim 18.

25. A plant or fungus, containing a nucleic acid molecule according to claim 20.

26. The plant or fungus of claim 25, which is an agricultural crop and/or an ornamental plant selected from
Zea mays, Gossypium spp., Capsicum spp., Solanum tuberosum, Solanum lycopersicum, Nicotiana tabacum, Phaseolus lunatus, Pisum sativum var. macrocarpon, Glycine max, Arachis hypogaea, Triticum aestivum, Avena sativa, Hordeum vulgare, Secale cereale, Malus domestica, Pyrus communis, Prunus spp., Ribes spp., and Vitis vinifera,
or an edible, medicinal or ornamental mushroom selected from
Agaricus bisporus, Pleurotus ostreatus, P. eryngii, Lentinula edodes, Hericium spp., Volvariella volvacea, Grifola frondosa, Ganoderma spp., Trametes spp, Auricularia polytricha, Flammulina velutipes, Lentinus sajor-caju, and Hypsizygus tessellatus,
or an entomopathogenic or mite-pathogenic fungus selected from
Beauveria bassiana, Hirsutella thompsonii, Isaria spp., Lecanicillium spp., Metarhizium spp., and Nomuraea spp.

27. The plant of claim 25, furthermore comprising Bacillus thuringiensis or Bacillus thuringiensis subspecies israelensis (Bti).

28. A method for pest control comprising contacting the pest with
the protein of claim 18 as (bio)insecticide.

29. The method according to claim 28 used to control mosquitoes.

30. The method according to claim 28 used against insect pests attacking crop plants and/or ornamental plants.

31. The method according to claim 28 used to control mites,
fungus gnats and fungus pests,
storage pests,
hygiene pests,
and/or
in crop protection.

32. A (bio)insecticide or (bio)pesticide composition comprising:
(a) a protein of claim 18, a nucleic acid molecule of claim 18, and
(b) excipient(s) and/or carrier.

33. The composition of claim 32, furthermore comprising one or more of the following:
methionine
temephos
Bacillus thuringiensis subspecies israelensis toxins (Bti toxins),
Lysinibacillus sphaericus powder SPH88,
chitin synthesis inhibitors, e.g. diflubenzuron, novaluron,
pyriproxyfen,
methoprene,
anethole,
cinnamaldehyde,
cinnamyl acetate,
Bacillus thuringiensis toxins (Bt toxins),
azadirachtin,
tebufenozide, and
malathion.

34. The composition of claim 32, which is formulated as a solution, a powder, granules, or a bait.

35. The nucleic acid molecule of claim 20, which encodes a protein selected from a protein comprising an amino acid sequence of SEQ ID NO: 1 or an amino acid sequence having at least 60% sequence identity to the amino acid sequence of SEQ ID NO: 1.

36. The host cell of claim 23, wherein the cell is a bacterial cell that is Escherichia coli,
a plant cell that is
Zea mays, Gossypium spp., Capsicum spp., Solanum tuberosum, Solanum lycopersicum, Nicotiana tabacum, Phaseolus lunatus, Pisum sativum var. macrocarpon, Glycine max, Arachis hypogaea, Triticum aestivum, Avena sativa, Hordeum vulgare, Secale cereale, Malus domestica, Pyrus communis, Prunus spp., Ribes spp., or Vitis vinifera,
or
a fungal cell that is
an edible, medicinal or ornamental mushroom or a yeast cell or an entomopathogenic or mite-pathogenic fungus that
Agaricus bisporus, Pleurotus ostreatus, P. eryngii, Lentinula edodes, Hericium spp., Volvariella volvacea, Grifola frondosa, Ganoderma spp., Trametes spp, Auricularia polytricha, Flammulina velutipes, Lentinus sajor-caju, Hypsizygus tessellatus,
Ustilago spp., Microbotryum spp., Xanthophyllomyces dendrorhous, Rhodotorula spp., Sporobolomyces spp., Mrakia spp.,
Beauveria bassiana, Hirsutella thompsonii, Isaria spp., Lecanicillium spp., Metarhizium spp., or Nomuraea spp.

37. The method of claim 31 used to control
Brennandania lambi, Tyrophagus putrescentiae, Panonychus ulmi, Tetranychus urticae, Sciaridae, Camptomyia corticalis, C. heterobia,
Bruchids, Caryedon serratus, Plodia interpunctella; Sitophilus spp. Curculionidae; Cephalonomia tarsalis, Bethylidae, Anisopteromalus calandrae,
cockroaches, ants, termites, bed bugs, slugs, blossom beetle, rape stem beetle, Aphids, Drosophila susukii, raspberry weevil, caterpillars, fungus gnats, European grape vine moth, stem borers, or European red mite.


SEQUENCE LISTING (34 sequences)

SEQ ID NO: 1 - protein, 156 aa, Agrocybe aegerita
MSESSTFTTAVVPEGEGVAPMAETVQYYNSYSDASIASCAFVDSGKDKIDKTKLVTYTSRLAASPAYQKVVGVGLKTAAGSIVPYVRLDMDNTGKGIHFNATKLSDSSAKLAAVLKTTVSMTEAQRTQLYMEYIKGIENRSAQFIWDWWRTGKAPA

SEQ ID NO: 2 - DNA, 471 bp, Agrocybe aegerita
atgtccgagtcctctaccttcaccactgcggtagtacctgaaggcgaaggagttgctccaatggcagagaccgtgcagtattacaactcctactctgacgcatccatcgcgtcttgcgcatttgtagactcggggaaggacaaaattgataagaccaagttggtcacgtacaccagccgcctcgccgcaagccccgcatatcagaaggtcgtcggcgtcggcctcaaaacggccgcgggctccatcgtgccctacgtccggctcgacatggacaacaccggcaagggcatccatttcaacgcgactaaactctccgacagttccgccaagctcgccgcggtgctcaagacgacggtgtccatgaccgaggcacagcgaactcaactctacatggagtatatcaagggcatcgagaatcggagtgcgcagtttatttgggactggtggaggacgggcaaggctccggcgtga

SEQ ID NO: 3 - protein, 151 aa, Agrocybe aegerita
MSDPSAPGLEEGTEISPMAETVQTFASYSEASVAACKWVNSGKTQIDPAQLILYKNTLPASPAYGKIVGVGLKFTAEVDFCRLDMDNTGKGIHFNAKQRDDQSKKLAAVIKPTVALSEAQRTQLYMEYIKGLENRSAQFIWEWWSTGKAPA

SEQ ID NO: 4 - DNA, 456 bp, Agrocybe aegerita
atgtctgacccatccgcacccggactcgaagagggcaccgaaatctcacccatggccgagacggtgcagaccttcgcgagctactccgaggcctccgttgccgcttgcaagtgggtcaactcgggaaagacccagattgacccagcccagctcatcctgtacaagaacaccctccccgcaagccccgcatacggcaaaatcgtcggcgtcggcctaaaattcaccgccgaggtcgacttctgccgactcgacatggacaacacaggcaagggcatccacttcaacgccaagcagcgggacgaccagtccaagaagctcgcggcggtgatcaagccgactgttgcgttgagcgaggcccagcgcacgcagctgtacatggagtatatcaaggggctcgagaacaggagtgcgcagtttatttgggagtggtggagcaccgggaaggcaccggcgtga

SEQ ID NO: 5 - DNA, 36 bp, Artificial Sequence
  Primer
ggcgcatatgtccgagtcctctaccttcaccactgc

SEQ ID NO: 6 - DNA, 29 bp, Artificial Sequence
  Primer
gtgcggccgctcacgccggagccttgccc

SEQ ID NO: 7 - DNA, 34 bp, Artificial Sequence
  Primer
caccaccaccacgagtcctctaccttcaccactg

SEQ ID NO: 8 - DNA, 34 bp, Artificial Sequence
  Primer
atgatgatgatgggacatatgtatatctccttct

SEQ ID NO: 9 - DNA, 25 bp, Artificial Sequence
  Primer
aagttggtcacggccaccagccgcc

SEQ ID NO: 10 - DNA, 26 bp, Artificial Sequence
  Primer
ggtcttatcaattttgtccttccccg

SEQ ID NO: 11 - DNA, 24 bp, Artificial Sequence
  Primer
gtcgcgctcgacatggacaacacc

SEQ ID NO: 12 - DNA, 24 bp, Artificial Sequence
  Primer
gtagggcacgatggagcccgcggc

SEQ ID NO: 13 - DNA, 24 bp, Artificial Sequence
  Primer
tgccctacgtccggctcgccatgg

SEQ ID NO: 14 - DNA, 24 bp, Artificial Sequence
  Primer
cgatggagcccgcggccgttttga

SEQ ID NO: 15 - DNA, 24 bp, Artificial Sequence
  Primer
gtccggctcgacatggccaacacc

SEQ ID NO: 16 - DNA, 24 bp, Artificial Sequence
  Primer
gtagggcacgatggagcccgcggc

SEQ ID NO: 17 - DNA, 20 bp, Artificial Sequence
  Primer
ggcaagggcatcgctttcaa

SEQ ID NO: 18 - DNA, 20 bp, Artificial Sequence
  Primer
ggtgttgtccatgtcgagcc

SEQ ID NO: 19 - DNA, 19 bp, Artificial Sequence
  Primer
agttccgccgcgctcgccg

SEQ ID NO: 20 - DNA, 25 bp, Artificial Sequence
  Primer
gtcggagagtttagtcgcgttgaaa

SEQ ID NO: 21 - DNA, 25 bp, Artificial Sequence
  Primer
ttcttttcgctactcagaatcgttg

SEQ ID NO: 22 - DNA, 20 bp, Artificial Sequence
  Primer
cagagctctcccaaccacag

SEQ ID NO: 23 - DNA, 24 bp, Artificial Sequence
  Primer
agatgcgtattctgatggttggtc

SEQ ID NO: 24 - DNA, 23 bp, Artificial Sequence
  Primer
cccacactgtgaatgagatgttc

SEQ ID NO: 25 - DNA, 21 bp, Artificial Sequence
  Primer
attcctacgatccttttgccg

SEQ ID NO: 26 - DNA, 23 bp, Artificial Sequence
  Primer
gatcatattgtttcgggagtcct

SEQ ID NO: 27 - DNA, 21 bp, Artificial Sequence
  Primer
tctgatctgactgtcggccaa

SEQ ID NO: 28 - DNA, 21 bp, Artificial Sequence
  Primer
atcctcgtccttatgctcctc

SEQ ID NO: 29 - DNA, 19 bp, Artificial Sequence
  Primer
aagccccgcatatcagaag

SEQ ID NO: 30 - DNA, 20 bp, Artificial Sequence
  Primer
ctgtcggagagtttagtcgc

SEQ ID NO: 31 - DNA, 21 bp, Artificial Sequence
  Primer
gaaagacccagattgacccag

SEQ ID NO: 32 - DNA, 19 bp, Artificial Sequence
  Primer
gtgaattttaggccgacgc

SEQ ID NO: 33 - DNA, 56 bp, Artificial Sequence
  Primer
ggggggcatatgagccatcatcatcatcaccaccaccacgacccgagcgcgccggg

SEQ ID NO: 34 - DNA, 37 bp, Artificial Sequence
  Primer
ggggggctcgagtgcggccgcttacgccggcgctttg